(19) United States
(12) Patent Application Publication
(10) Pub. No.: US 2008/0152648 A1
(43) Pub. Date: Jun. 26, 2008

(54) Compositions and methods for enhancing an adjuvant

(75) Inventors: Anke Kretz-Rommel, San Diego, CA (US); Katherine S. Bowdish, Del Mar, CA (US); Paul J. Tacken, Nijmegen (NL); Carl Gustav Figdor, Den Bosch (NL); Susan Faas McKnight, Old Lyme, CT (US)

Correspondence Address: ROPES & GRAY LLP, PATENT DOCKETING 39/41, ONE INTERNATIONAL PLACE, BOSTON, MA, 02110-2624, US

(73) Assignees: Alexion Pharmaceuticals, Inc., Cheshire, CT (US); Stichting Katholieke Univesiteit, Nijmegen (NL)

(21) Appl. No.: 11/904,045

(22) Filed: Sep. 24, 2007

Related U.S. Application Data
(60) Provisional application No. 60/847,407, filed on Sep. 26, 2006.

(51) Int. Cl. A61K 39/00

(52) U.S. Cl. 424/133.1; 424/172.1

(57) ABSTRACT

Provided herein are compositions and methods for enhancing an adjuvant effect. The methods involve targeting an adjuvant to an antigen presenting cell (APC) using a compound that binds to a cell surface marker of an APC. Such methods are useful for stimulating an immune response in an animal, such as a human. Also provided are compositions that target an antigen and an adjuvant to an APC.

### RELATED APPLICATIONS

This application claims the benefit of U.S. Patent Application No. 60/847,407, filed Sep. 26, 2006, which application is hereby incorporated by reference in its entirety.

### BACKGROUND

The mammalian immune system provides a means for the recognition and elimination of tumor cells, other pathogenic cells, and invading foreign pathogens. While the immune system normally provides a strong line of defense, there are still many instances where cancer cells, other pathogenic cells, or infectious agents evade a host immune response and proliferate or persist with concomitant host pathogenicity. Chemotherapeutic agents and radiation therapies have been developed to eliminate replicating neoplasms. However, most, if not all, of the currently available chemotherapeutic agents and radiation therapy regimens have adverse side effects because they work not only to destroy cancer cells, but they also affect normal host cells, such as cells of the hematopoietic system. Furthermore, chemotherapeutic agents have limited efficacy in instances where host drug resistance is developed.

Foreign pathogens can also proliferate in a host by evading a competent immune response or where the host immune system has been compromised by drug therapies or by other health problems. Although many therapeutic compounds have been developed, many pathogens are or have become resistant to such therapeutics. The capacity of cancer cells and infectious organisms to develop resistance to therapeutic agents, and the adverse side effects of the currently available anticancer drugs, highlight the need for the development of new therapies specific for pathogenic cell populations and with reduced host toxicity.

The immune system may exhibit both specific and nonspecific immunity with specific immunity being mediated by B and T lymphocytes which display receptors on their surfaces for specific antigens. The specific immune response may involve humoral immunity (i.e., B cell activation with the production of antibodies), and cell-mediated immunity (i.e., activation of T cells, such as cytotoxic T lymphocytes, helper T lymphocytes, including T_{H1} and T_{H2} cells, and antigen-presenting cells). T_{H1} responses elicit complement fixing antibodies, activation of cytotoxic T lymphocytes, and strong delayed-type hypersensitivity reactions and are associated with the production of IL-2, IL-12, TNF, lymphotoxin, and gamma-interferon. T_{H2} responses are associated with the production of IgE, and IL-4, IL-5, IL-6, and IL-10. A specific immune response involves not only specificity, but also memory so that immune cells previously exposed to an antigen can rapidly respond to that same antigen upon future exposure to the antigen.

Adjuvants are compounds or materials that stimulate immune responses, for example, by augmenting the immunogenicity of an antigen. Adjuvants can act either nonspecifically, stimulating the immune response to a wide variety of antigens, or specifically (i.e., stimulating the immune response in an antigen-specific manner). Adjuvants that enhance specific immunity can act by stimulating the cell-mediated immune response or the humoral response or both. Adjuvants that stimulate the cell-mediated immune response can bias the immune response towards a T_{H1} or a T_{H2} response. Adjuvants that stimulate the humoral immune response can induce the production of an antibody isotype profile that differs depending on the adjuvant used. In this regard, different adjuvants can stimulate the production of different antibody isotypes, different levels of antibodies of each isotype, and can stimulate the production of antibodies with differing affinities, resulting in divergent antibody populations depending on the adjuvant used.

Development of safe and effective compositions and methods for stimulating an immune response is of great importance. In particular, a need exists for methods to stimulate an immune response to tumor associated antigens and pathogen associated antigens without the need to first identify such antigens or individualize the treatment for a specific antigen.

### SUMMARY

Provided herein are methods and compositions for enhancing an adjuvant effect. Also provided are methods for treating an animal, such as a human, in need of immune stimulation.

In one aspect, the invention provides a method for enhancing the effect of an adjuvant, comprising administering to an animal an agent comprising an adjuvant attached to a compound that binds to a cell surface marker of an antigen presenting cell (APC), thereby targeting the adjuvant to an antigen presenting cell (APC) and enhancing the effect of the adjuvant by stimulating an immune response to an antigen other than the compound or the adjuvant. The adjuvant may be covalently attached to the compound that binds to the APC cell surface marker.

In certain embodiments, the compound may be an antibody that binds to a cell surface marker of an APC. Such antibody may be an antibody fragment, an internalizing antibody, a monoclonal antibody, or a polyclonal antibody.

In certain embodiments, the APC may be a dendritic cell. When the APC is a dendritic cell, the cell surface marker may be a C-type lectin, such as, for example, DC-SIGN. In such instances, the compound that binds to DC-SIGN may be, for example, a mannose carbohydrate, a fucose carbohydrate, a plant lectin, an antibiotic, a sugar, a protein, or an antibody. In an exemplary embodiment, the compound that binds to DC-SIGN is an antibody, such as, for example, a monoclonal antibody.

In certain embodiments, the adjuvant may be, for example, a mineral salt, a small molecule, a saponin, a polysaccharide, a lipid, a nucleic acid, a protein or a peptide. In an exemplary embodiment, the adjuvant is a protein, such as, for example, keyhole limpet hemocyanin (KLH) or bacillus calmette guerin (BCG).

In certain embodiments, the animal is a human.

In certain embodiments, the adjuvant stimulates a naive immune response.

In certain embodiments, the animal was not previously vaccinated with said adjuvant.

In certain embodiments, the agent further comprises an antigen covalently attached to said compound.

In another aspect, the invention provides a method for stimulating an immune response in an animal in need thereof, comprising administering to said animal an agent comprising an adjuvant attached to a compound that binds to a cell surface marker of an antigen presenting cell (APC), wherein said agent stimulates an immune response to an antigen other than the compound or the adjuvant (although a response to the compound and/or adjuvant may also occur). The adjuvant may be covalently attached to the compound that binds to a cell surface marker of an APC.

In certain embodiments, the agent stimulates an immune response to a tumor associated antigen or an antigen associated with a pathogen infection.

In another aspect, the invention provides a method for stimulating an immune response in an animal, comprising administering to said animal an APC targeting agent comprising a DC-SIGN specific antibody covalently linked to an adjuvant, wherein said APC targeting agent stimulates an immune response to an antigen other than the DC-SIGN specific antibody or the adjuvant, and wherein said animal was not previously vaccinated with said adjuvant. It is to be noted that in addition to the immune response to said antigen an immune response to the antibody and/or adjuvant may also occur

In another aspect, the invention provides an immunostimulatory agent comprising: a compound that binds to a cell surface marker of an antigen presenting cell (APC), an adjuvant, and an antigen, wherein the compound, adjuvant and antigen are attached. In certain embodiments, the adjuvant, antigen, and compound may be covalently attached. In various embodiments, the adjuvant, antigen and compound may be attached in any order.

In certain embodiments, the antigen may be, for example, a tumor associated antigen or an antigen from a pathogen.

In certain embodiments, the adjuvant may be, for example, a mineral salt, a small molecule, a saponin, a polysaccharide, a lipid, a nucleic acid, a protein or a peptide. In an exemplary embodiment, the adjuvant is a protein, such as, for example, keyhole limpet hemocyanin (KLH) or bacillus calmette guerin (BCG).

In certain embodiments, the compound is an antibody that binds to a cell surface marker of an APC. Such an antibody may be, for example, an antibody fragment, an internalizing antibody, a monoclonal antibody or a polyclonal antibody. When the compound is an antibody, the antigen may be attached to the C-terminus of a heavy chain constant region. Alternatively, the antigen may be incorporated into a complementarity determining region (CDR) of the antibody or into the constant region of the antibody.

In certain embodiments, the compound binds to a cell surface marker of a dendritic cell. Exemplary dendritic cell surface markers are C-type lectins, such as, for example, DC-SIGN. Examples of compounds that bind to DC-SIGN include, for example, a mannose carbohydrate, a fucose carbohydrate, a plant lectin, an antibiotic, a sugar, a protein, or an antibody. In an exemplary embodiment, a compound that binds to DC-SIGN is an antibody, such as, for example, a monoclonal antibody.

In another aspect, the invention provides an immunostimulatory composition comprising a compound that binds to a cell surface marker of an antigen presenting cell (APC), an adjuvant and an antigen, wherein at least one of the adjuvant or the antigen is attached to said compound.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (J. Woodward ed., IRL Press, 1985); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Hogan, B., Costantini, F. and Lacy, E., Cold Spring Harbor Laboratory Press, Cold Spring Harbor, N.Y., 1986).

Other features and advantages of the invention will be apparent from the following detailed description and from the claims.

The appended claims are incorporated into this section by reference.

### BRIEF DESCRIPTION OF THE FIGURES

The foregoing and other features and advantages of the present invention will be more fully understood from the following detailed description of illustrative embodiments taken in conjunction with the accompanying drawings in which:

FIGS. 1A-1D. Cord blood grafted mice are capable of mounting a proliferative response to KLH after immunizations with KLH in Titermax or DC-SIGN targeted immunization with D1V1-KLH. FIG. 1A: Four grafted mice at 15 weeks of age were injected with 50 μg KLH in Titermax, half the amount s.c., the other half i.p. Two immunized mice (average shown) and one unimmunized mouse were sacrificed on Day 12, and spleen cells were isolated. Proliferation in response to KLH was tested by determining ³H-thymidine incorporation after 5 days in culture. FIG. 1B: Two mice received a second immunization 3 weeks after the first. Spleens were taken 12 days later. Proliferation of splenocytes to KLH was evaluated by ³H-thymidine incorporation. FIG. 1C: Six grafted mice were immunized i.v. with either 100 μg D1V1-KLH or 100 μg 5G1.1-KLH. Two additional grafted mice were also injected with 100 μg KLH and Titermax s.c. Spleens were harvested nine days (FIG. 1C) or, FIG. 1D, 14 days (FIG. 1D) later and proliferation response to KLH was evaluated by ³H-thymidine incorporation assay. Data represent mean of 3-6 replicates ±S.D.

FIG. 2. CD40 antibodies do not alter immune responses to antigens targeted to DCs via DC-SIGN antibodies. 100 μg D1V1-KLH with or without 90 μg anti-human CD40 were injected i.v. into cord blood engrafted Rag2^{-/-}γc^{-/-} mouse. Nine days after immunization, the spleens were taken out and splenocytes were stimulated with various concentrations of KLH for 5 days. Proliferation of splenocytes was assessed by ³H-thymidine incorporation. Data represent mean of 3-6 replicates ±S.D.

FIGS. 3A-3C. Delivery of tetanus toxoid peptide by a different DC-SIGN antibody (E10-TT) also elicits an immune response. FIG. 3A: Cord blood grafted mice at 15 weeks of age were immunized with 50 μg tetanus toxoid (TT) in Titermax, half the amount s.c., the other half i.p. Some mice were sacrificed and spleens were isolated 9 days after immunization (FIG. 3A) or some mice were sacrificed after a second immunization and spleens were isolated 9 days after immunization (FIG. 3B). Proliferation in response to TT was determined by ³H-thymidine incorporation after 5 days in culture. FIG. 3C: 3 nmol E10-TT or 3 nmol E10 mixed with 3 nmol TT was injected s.c. into 5 Rag2^{-/-}γc^{-/-} mice each. After 9 days, spleens were taken and splenocytes were stimulated with either E10 or TT. 1 μCi ³H-thymidine was added into each well 4 days later. The cells were harvested in 24 hours. Values represent mean of 3-6 wells ±S.D.

FIG. 4. Antigen targeting to DCs by DC-SIGN antibodies does not require dimerization to elicit a stimulatory immune response. Either 3.3 nmol scFv-RR-TT or D1V1scFv mixed with TT dipeptide were injected i.v. into 3 grafted mice each. One mouse was immunized s.c. with dipeptide in Titermax. Spleens were harvested after 9 days and the cells stimulated with PHA, dipeptide or medium alone. ³H-thymidine was added after 4 days, and cells were harvested and counts determined the following day. Values represent mean of 3-6 wells ±S.D.

FIGS. 5A-5D. Adjuvant effect of D1V-KLH in RAJI/Fludarabine-treated RAJI/iDC/hPBL model: NOD/SCID mice were injected with 4 million RAJI cells (FIG. 5A, FIG. 5C) or 3 million RAJI cells and 2 million fludarabine treated RAJI cells (fdRAJI) (FIG. 5B, FIG. 5D), 30000 immature DCs, and three million hPBLs. Equimolar amounts of D1V1-KLH (40 μg), control antibody 5G1.1-G2/G4 (40 μg), or KLH (10 μg) were injected s.c. with the cell mixture. Antibodies or KLH were subsequently administered 2 more times, 1 week apart using 200 μg D1V1-KLH, or 200 μg control antibody, or 50 μg KLH. Data represent mean ±SEM of tumor volumes from 10 mice of each group. *significant values (p<0.01) compared with control antibody treated group as calculated by Student's t-test. ** highly significant values (p<0.005) compared with control antibody treated group.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The terms “comprise” and “comprising” are used in the inclusive, open sense, meaning that additional elements may be included.

The term “including” is used to mean “including but not limited to”. “Including” and “including but not limited to” are used interchangeably.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). IgG, IgA and IgD isotypes have a “hinge region” which is an amino acid sequence of from about 10-60 amino acids that confers flexibility on the immunoglobulin molecule. The variable regions of each light/heavy chain pair form the antibody binding site such that an intact immunoglobulin has two binding sites. Immunoglobulins may be organized into higher order structures. IgA is generally a dimer of two tetramers. IgM is generally a pentamer of five tetramers.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin, or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, chimeric antibodies, minibodies, diabodies, triabodies, tetrabodies and polypeptides that contain at least a portion of an immunoglobulin that is sufficient to confer specific antigen binding to the polypeptide.

An Fab fragment is a monovalent fragment consisting of the VL, VH, CL and CH1 domains; a F(ab′)₂ fragment is a bivalent fragment comprising two Fab fragments linked by a disulfide bridge at the hinge region; an Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which VL and VH regions are paired to form a monovalent molecule via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent or bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently. A minibody is a bivalent or bispecific antibody in which two scFv monomers are joined by two constant domains (see e.g., Hudson, P. J. and Sourisu, C., Nature Medicine 9: 129-134 (2003)).

An antibody may have one or more binding sites. If there is more than one binding site, the binding sites may be identical to one another or may be different. For instance, a naturally-occurring immunoglobulin has two identical binding sites (e.g., bivalent), a single-chain antibody or Fab fragment may have one or two binding sites, while a “bispecific” or “bifunctional” antibody has two different binding sites.

The term “human antibody” includes all antibodies that have one or more variable and constant regions derived from human immunoglobulin sequences. In a preferred embodiment, all of the variable and constant domains are derived from human immunoglobulin sequences (a fully human antibody). These antibodies may be prepared in a variety of ways, as described below.

The term “hinge” or “hinge region,” as used herein, refers to a region of the heavy chain that comprises amino acid residues 224 to 251 (Kabat numbering scheme). This region encompasses the genetic hinge (e.g., amino acid residues 224-243 using the Kabat numbering scheme) as well as amino acid residues C-terminal to the genetic hinge that are structurally flexible (see e.g., Burton D R, Molecular Genetics of Immunoglobulin, Chapter 1, Calabi, F. and Neuberger, M. S., eds; Elsevier Science Publishers B.V. (1987)).

The term “chimeric antibody” refers to an antibody that contains one or more regions from one antibody and one or more regions from one or more other antibodies.

The term “tumor-associated antigen” refers to a polypeptide which is preferably presented by tumor cells and thus allows a distinction between tumor cells and non-tumor cells. Tumor associated antigens are proteins expressed inside or on the surface of tumor cells which are putative targets for immune responses. They often differ from normal cellular counterparts by mutations, deletions, different levels of expression, changes in secondary modifications or expression in other stages of development. The proteins are preferably expressed on the cellular surface and, in addition, presented as processed peptides on the tumor cell surface by MHC class I molecules. Examples of tumor-associated antigens include, for example, CA125, CA19-9, CA15-3, D97, gp100, CD20, CD21, TAG-72, EGF receptor, Epithelial cell adhesion molecule (Ep-CAM), Carcinoembryonic antigen (CEA), Prostate specific antigen (PSA), PMSA, CDCP1, CD26, Hepsin, HGF (hepatocyte growth factor), Met, CAIX(G250), EphhB4 (Ephrin type-B receptor 4), EGFR1, EGFR2, PDGF, VEGFR, DPP6, syndecan 1, IGFBP2 (Human insulin-like growth factor binding protein 2), CD3, CD28, CTL4, VEGF, Her2/Neu receptor, tyrosinase, MAGE 1, MAGE 3, MART, BAGE, TRP-1, CA 50, CA 72-4, MUC 1, NSE (neuron specific enolase), α-fetoprotein (AFP), SSC (squamous cell carcinoma antigen), BRCA-1, BRCA-2 and hCG.

The term “therapeutically effective amount” refers to that amount of an APC targeting agent, drug or other molecule which is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

### 2. Compositions for Enhancing an Adjuvant Effect

Provided herein are APC targeting agents for enhancing an adjuvant effect that may be used, for example, in accordance with the methods described herein. Such agents comprise an adjuvant covalently linked to a compound that binds to a cell surface protein on an APC. Such agents may optionally comprise an additional component, such as, for example, an antigen. Compositions comprising an APC targeting agent and a pharmaceutically acceptable carrier are also provided.

The APC targeting agents described herein comprise a compound that targets an adjuvant to a cell surface protein of an APC. In an exemplary embodiment, the compound targets an adjuvant to an internalizing receptor on an APC. A variety of cells can function as APCs. The distinguishing feature of these cells is their ability to express class II MHC molecules and to deliver a co-stimulatory signal. Three cell types are classified as professional APCs: dendritic cells, macrophages and B lymphocytes. In an exemplary embodiment, a compound that specifically binds to an APC cell surface protein is an antibody, for example, an anti-MHC II antibody, or an antibody to the cell surface markers described below.

Cell surface markers specific to dendritic cells include, for example, CD83, CD205/DEC-205, CD197/CCR7 and CD209/DC-SIGN. Compounds that specifically bind to such DC cell surface specific markers may be used to target an adjuvant to a DC. In an exemplary embodiment, a compound may be used to target an adjuvant to an internalizing receptor on the surface of a DC, such as, for example, the DEC-205, DC-SIGN or CCR7 receptors. Compounds that can bind to a DC cell surface marker may be, for example, a carbohydrate, a lectin, an antibiotic, a sugar, a protein, a peptide, or an antibody.

In certain embodiments, a compound may be targeted to DC-SIGN. DC-SIGN is a C-type lectin which binds to ICAM receptors on T cells and facilitates adhesion of HIV to dendritic cells (see e.g., U.S. Patent Publication No. 2005/0118168). Compounds that can bind to DC-SIGN include, for example, mannose carbohydrates such as mannan and D-mannose; fucose carbohydrates such as L-fucose; plant lectins such as concanavalin A; antibiotics such as pradimicin A; sugars such as N-acetyl-D-glucosamine and galactose; as well as suitable peptidomimetic compounds and small drug molecules, which can for instance be identified using phage display techniques. Furthermore, proteins such as gp120 and analogs or fragments thereof that maintain DC-SIGN binding activity may be used, as well as isolated ICAM-receptors and analogs thereof, including DC-SIGN binding parts or fragments thereof.

In certain embodiments, a compound may be an antibody targeted to a DC specific cell surface protein, e.g., CD205/DEC-205, CD197/CCR7 and CD209/DC-SIGN. Such antibodies may be purchased commercially, or produced using standard techniques, as outlined further herein. In an exemplary embodiment, a compound for targeting an adjuvant to an APC is a anti-DC-SIGN antibody, such as, for example, AZN-D1 or AZN-D2 (see e.g., U.S. Patent Publication No. 2005/0118168). In an exemplary embodiment, a compound may be an antibody targeted to an internalizing receptor on the surface of a DC, such as, for example, an antibody that binds to DEC-205, DC-SIGN or CCR7.

Cell surface markers specific to B lymphocytes include, for example, CD19, CD20, CD21, CD22, CD32, CD79α,β, CD83, CD138, CD139, CD179a,b, and CD180. Compounds that specifically bind to such B lymphocyte specific cell surface markers may be used to target an adjuvant to a B lymphocyte. Compounds that can bind to a B lymphocyte cell surface marker may be, for example, a protein, a peptide or an antibody. In an exemplary embodiment, a compound may be an antibody targeted to an internalizing receptor on the surface of a B lymphocyte, such as, for example, a protein, a peptide or an antibody that binds to Cd19, CD20, CD21, CD22, CD32, or CD79.

Cell surface markers specific to macrophages include, for example, CD64, CD169, CD170 and CD206. Compounds that specifically bind to such macrophage specific cell surface markers may be used to target an adjuvant to a macrophage. Compounds that can bind to a macrophage cell surface marker may be, for example, a protein, a peptide or an antibody. In an exemplary embodiment, a compound may be an antibody targeted to an internalizing receptor on the surface of a macrophage, such as, for example, a protein, a peptide or an antibody that binds to CD64 or CD206.

Antibodies for use in targeting an adjuvant to an APC may be IgG, IgM, IgE, IgA or IgD molecules. Such antibodies may comprise a constant region, or a portion thereof, from any type of antibody isotype, including, for example, IgG (including IgG1, IgG2, IgG3, and IgG4), IgM, IgE, IgA or IgD, or a hybrid constant region, or a portion thereof, such as a G2/G4 hybrid constant region (see e.g., Burton D R and Woof J M, Adv. Immun. 51: 1-18 (1992); Canfield S M and Morrison S L, J. Exp. Med. 173: 1483-1491 (1991); Mueller J P, et al., Mol. Immunol. 34(6): 441-452 (1997)).

In certain embodiments, chimeric, humanized or primatized (CDR-grafted) antibodies, antibody fragments, as well as chimeric or CDR-grafted antibody fragments, comprising portions derived from different species, may be used for targeting an adjuvant to an APC. Exemplary antibody fragments include, for example, Fab, Fab′, F(ab′)₂ or minibodies, or Fv, scFv, diabodies and triabodies fused to at least a portion of a heavy chain and/or light chain constant region. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023; Boss et al., U.S. Pat. No. 4,816,397; Boss et al., European Patent No. 0,120,694; Neuberger, M. S. et al., WO 86/01533; Neuberger, M. S. et al., European Patent No. 0,194,276 B1; Winter, U.S. Pat. No. 5,225,539; and Winter, European Patent No. 0,239,400 B1. See also, Newman, R. et al., BioTechnology 10: 1455-1460 (1992), regarding primatized antibody. See, e.g., Ladner et al., U.S. Pat. No. 4,946,778; and Bird, R. E. et al., Science 242: 423-426 (1988)), regarding single chain antibodies.

In addition, functional fragments of antibodies, including fragments of chimeric, humanized, primatized or single chain antibodies can also be used to target an adjuvant to an APC. Functional antibody fragments refer to fragments that retain at least one binding function and/or modulation function of the full-length antibody from which they are derived. Preferred functional fragments retain an antigen binding function of a corresponding full-length antibody.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293. A humanized antibody may comprise portions of immunoglobulins of different origin, wherein optionally at least one portion is of human origin. Accordingly, a humanized immunoglobulin having binding specificity for a desired epitope, said immunoglobulin comprising an antigen binding region of nonhuman origin (e.g., rodent) and at least a portion of an immunoglobulin of human origin (e.g., a human framework region, a human constant region or portion thereof) may be used to target an adjuvant to an APC. For example, the humanized antibody can comprise portions derived from an immunoglobulin of nonhuman origin with the requisite specificity, such as a mouse, and from immunoglobulin sequences of human origin (e.g., a chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain).

In certain embodiments, antibodies useful as APC targeting compounds may be internalizing antibodies, e.g., antibodies that are taken up by a cell bound by the antibody.

The APC targeting agents described herein also comprise an adjuvant. The adjuvant is attached (e.g., covalently) to a compound that specifically binds to an APC cell surface molecule thereby targeting the adjuvant to an APC and enhancing the adjuvant effect. A wide variety of adjuvants may be used in accordance with the agents described herein. In exemplary embodiments, adjuvants that are suitable for administration to humans may be selected. Examples of the adjuvants that may be used in accordance with the methods and compositions described herein include, but are not limited to, a mineral salt, a saponin, a polysaccharide, a lipid, a lipopolysaccharide (endotoxin), a nucleic acid or a protein.

In certain embodiments, an adjuvant may be a mineral salt. Examples of mineral salt adjuvants include, but are not limited to, aluminum salts, aluminum phosphate (e.g., HCl Biosector Elsenbakken 23, DK-3600 Fredrikssund, Denmark), calcium phosphate (e.g., Superfos Biosector Als Frydenlundsvej 30, 2950 Vedback, Denmark), aluminum hydroxide (Alhydrogel), aluminum hydroxide in combination with gamma insulin (Algammulin), amorphous aluminum hydroxyphosphate (Adju-Phos), and deoxycholic acid-aluminum hydroxide complex (DOC/Alum).

In another embodiment, an adjuvant may be a synthetic imidazoquinoline such as imiquimod (S-26308, R-837) (Harrison et al., Vaccine 19: 1820-1826 (2001)) or resiquimod (S-28463, R-848) (Vasilakos et al., Cellular Immunology 204: 64-74 (2000)).

In certain embodiments, an adjuvant may be a saponin. A saponin encompasses natural and synthetic glycosidic triterpenoid compounds and pharmaceutically acceptable salts, derivatives, mimetics (e.g., isotucaresol and its derivatives) and/or biologically active fragments thereof, which possess adjuvant activity. Exemplary saponins include, for example, Quillaja saponin, QS-7, QS-17, QS-18, QS-21, Quil-A (see, e.g., U.S. Pat. No. 5,057,540), and GSK-1 (ginseng saponin).

In certain embodiments, an adjuvant may be a nucleic acid. Examples of nucleic acid adjuvants include, but are not limited to, CpG, polyadenylic acid/polyuridylic acid, and Loxorbine (7-allyl-8-oxoguanosine) (see e.g., U.S. Pat. No. 6,406,705).

In other embodiments, an adjuvant may be a protein or protein fragment. Examples of protein adjuvants include hemocyanins, hemoerythrins, serum proteins, cytokines, macrophage inflammatory proteins, bacterial antigens, yeast antigens, mammalian polypeptides, and superantigens.

In an exemplary embodiment, adjuvants may be hemocyanins and hemoerythrins. An exemplary hemocyanin is from keyhole limpet (KLH), although other molluscan and arthropod hemocyanins and hemoerythrins may be employed.

In another exemplary embodiment, a protein adjuvant may be bacillus calmette guerin (BCG).

In another embodiment, a protein adjuvant may be a serum protein, such as, for example, complement factor C3d. C3d is a 16 amino acid peptide (See, e.g., Fearon et al., 1998, Semin. Immunol. 10: 355-61; Nagar et al., 1998, Science; 280(5367):1277-81, Ross et al. 2000, Nature Immunol., Vol. 1(2)) which is available commercially (e.g., Sigma Chemical Company Cat. C 1547).

In other embodiments, a protein adjuvant may be a cytokine. Examples of cytokines that may be used in the compositions of the invention include, but are not limited to, interferons (e.g., interferon-gamma), interleukins (e.g., interleukin-2 (IL-2), interleukin-4 (IL-4), interleukin-6 (IL-6), interleukin-7 (IL-7), interleukin-12 (IL-12), interleukin-15 (IL-15)), colony stimulating factors, e.g., macrophage colony stimulating factors (M-CSF); G-CSF, GM-CSF, tumor necrosis factor (TNF), IL-1 and MIP-3a.

In certain embodiments, a protein adjuvant may be a macrophage inflammatory protein (MIP), or fragments thereof. MIPs are proteins that are produced by certain mammalian cells, for example, macrophages and lymphocytes, in response to stimuli, such as gram-negative bacteria, lipopolysaccharide and concanavalin A. An exemplary MIP is macrophage inflammatory protein 3 (MIP-3) (Genbank Accession No. P55773).

In another embodiment, a protein adjuvant may be a bacterial or yeast antigen. Examples of suitable bacterial or yeast antigens include, for example, muramyl peptides (such as, Immther™, theramide (MDP derivative), DTP-N-GDP, GMDP (GERBU adjuvant), MPC-026, MTP-PE, murametide, and murapalmitine); MPL derivatives (such as, MPL-A, MPL-SE, 3D-MLA, and SBAS-2); mannon, N-acetyl-muramyl-L-threonyl-D-isoglutamine (thr-MDP), N-acetyl-nor-muramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine (MTP-PE). Such agents are commercially available, for example, MPL-A may be obtained from ICN Chemical Company (Cat # 150012) and Immther™ may be obtained from Dor Pharma Inc.

In other embodiments, a protein adjuvant may be a mammalian peptide. Examples of mammalian peptides that may be used as adjuvants include, for example, melanoma peptide 946, neutrophil chemo-attractant peptide, and elastin repeating peptide.

In certain embodiments, an adjuvant may be a superantigen. Superantigens may be particularly useful for generating or enhancing the immune response against intracellular antigens, including intracellular tumor antigens. Superantigens are bacterial products that stimulate a greater proportion of T lymphocytes than peptide antigens without a requirement for antigen processing (Mooney et. al., (1994), Mol. Immunol. 31: 675-681). Superantigens include Staphylococcus exoproteins, such as the alpha, beta, gamma and delta enterotoxins from S. aureus and S. epidermidis, and the alpha, beta, gamma and delta E. coli exotoxins, and other membrane proteins and toxins from bacteria such as Clostridium perfringens and Streptococcus pyogenes.

In other embodiments, an adjuvant may be a polysaccharide. Various polysaccharide adjuvants may also be used, such as, for example, pneumococcal polysaccharide adjuvants (see e.g., Yin et al., (1989) J. Biological Response Modifiers 8: 190-205). Polyamine varieties of polysaccharides, such as chitin and chitosan, and deacetylated chitin, may also be used.

In other embodiments, an adjuvant may be a lipopolysaccharide (endotoxin). This class of adjuvants may be exemplified by lipid A, which may be used in animals, and detoxified endotoxins, which may be used in animals and humans. Detoxified and refined endotoxins, and combinations thereof, are described in U.S. Pat. Nos. 4,866,034; 4,435,386; 4,505,899; 4,436,727; 4,436,728; 4,505,900.

In another embodiment, adjuvants may be teichoic acids from Gram negative cells. These include the lipoteichoic acids (LTA), ribitol teichoic acids (RTA) and glycerol teichoic acid (GTA). Active forms of their synthetic counterparts may also be employed (Takada et al., (1995) Infection and Immunity 63: 57-65)).

BCG and BCG-cell wall skeleton (CWS) may also be used as adjuvants, with or without trehalose dimycolate. Trehalose dimycolate may be used itself. Trehalose dimycolate may be prepared as described in U.S. Pat. No. 4,579,945.

Adjuvants described herein are commercially available, or can be obtained using conventional methods well-known in the art.

Adjuvants may be covalently attached to a compound that binds to an APC cell surface molecule by a variety of methods known to those skilled in the art. For example, the adjuvant may be attached to the APC targeting compound using a chemical, peptide or oligomeric linker. In an exemplary embodiment, an APC targeting agent comprises an antibody directed to an APC cell surface molecule and an adjuvant. In such embodiments, the adjuvant is preferably attached to the antibody in a manner that does not affect the antigen binding activity of the antibody. For example, the adjuvant may be attached to the C-terminus of the constant region of the heavy and/or light chain using a chemical linker, peptide linker, oligomeric linker or using recombinant techniques. In an exemplary embodiment, an adjuvant attached to an antibody may be a protein adjuvant. In such embodiments, the adjuvant may be attached to the C-terminus of the heavy and/or light chain constant region by a peptide or chemical linker. The protein adjuvant may also be attached to the C-terminus of the constant region of the antibody heavy and/or light chain using recombinant techniques, e.g., a nucleic acid encoding the adjuvant may be appropriately joined with a nucleic acid encoding an antibody heavy or light chain and the adjuvant/antibody molecule expressed as a fusion.

In certain embodiments, an adjuvant may be attached to the C-terminus of the constant region of the heavy or light chain of an antibody by a cleavable peptide linker. The peptide linker may comprise for example two, three, four, five, or more, small amino acid residues, such as glycine or serine. Additionally, the linkers may comprise one or more amino acids that cause proteasomal cleavage (typically lysine or arginine) located near or directly adjacent to the N-terminus of the polypeptide. Exemplary linkers include, for example, peptides having the sequence GGXₙ (SEQ ID NO: 1), GGGXₙ (SEQ ID NO: 2), GGGGXₙ (SEQ ID NO: 3), GGGSXₙ (SEQ ID NO: 4), or GGGSGGGSXₙ (SEQ ID NO: 5), wherein X is lysine or arginine and n is 1-5.

In certain embodiments, it may be desirable to encapsulate the adjuvant in a liposomal carrier in order to attach the adjuvant to the APC targeting compound. Such techniques may be desirable, for example, when the adjuvant is a small molecule such as a mineral salt.

In certain embodiments, two or more different adjuvants may be covalently attached to an APC targeting compound. When using a combination of two or more adjuvants, the adjuvants may be of the same or different type. For example, it may be desirable to use two different protein adjuvants, two different oligonucleotide adjuvants, two different polysaccharide adjuvants, a protein adjuvant and an oligonucleotide adjuvant, a protein adjuvant and a polysaccharide adjuvant, etc.

In certain embodiments, the APC targeting agent may further comprise an additional component. In an exemplary embodiment, an APC targeting agent comprises an adjuvant and an antigen covalently attached to an APC targeting compound. The adjuvant is covalently attached to the APC targeting compound and the antigen may be covalently attached to either the APC targeting compound or to the adjuvant. In an exemplary embodiment, the antigen is different from the adjuvant.

Antigens may be attached to the APC targeting compound (e.g., directly to the APC targeting compound or to the adjuvant) using a chemical linker, a peptide linker, an oligomeric linker or using recombinant techniques as described above for attaching an adjuvant to the APC targeting carrier. Additionally, a peptide antigen may be incorporated into an antibody APC targeting compound by introducing the amino acid sequence of the antigen into a region of the antibody, e.g., a CDR region or constant region (see e.g., U.S. Patent Publication No. 2004/0253242). In an exemplary embodiment, a peptide antigen is incorporated into or near the hinge region of an antibody. The antigen sequence may be incorporated into the hinge region itself, at the junction between the N-terminus of the hinge region and the upstream region of the heavy chain, or at the junction between the C-terminus of the hinge region and the downstream region of the heavy chain. The antigen sequence may optionally be flanked by proteasomal cleavage sites. An antigen sequence may be incorporated into an antibody molecule by inserting the peptide into the antibody (e.g., the amino acid sequence is added to the sequence of the antibody). Alternatively, an antigen sequence may be incorporated into an antibody by replacing a portion of the antibody sequence with the introduced sequence. When incorporating an amino acid sequence by replacement, the length of the amino acid sequence being introduced may be the same size, larger or smaller than the antibody sequence being replaced (e.g., a sequence of 10 amino acids to be incorporated may replace a region of sequence on the antibody molecule that is 5, 10, or 15 amino acids in length). In an exemplary embodiment, the amino acid sequence is the same length as the region of the antibody sequence being replaced such that the overall size of the antibody molecule is maintained. An antigen sequence may be incorporated into the antibody at a region having a high degree of amino acid sequence identity with the sequence being incorporated.

Antigens included in an APC targeting agent may be any antigen for which it is desired to stimulate an immune response. For example, antigens may be derived from a pathogen or from a tumor cell, such that administration of the APC targeting agent to a subject will generate an immune response to said antigen. In an exemplary embodiment, an antigen may be a tumor associated antigen or an antigen associated with a pathogen.

Exemplary tumor associated antigens include the following: gp100, tyrosinase, MAGE-1, MAGE-3, MART, BAGE, and TRP-1 which are associated with melanoma; CEA (carcino embryonic antigen), CA 19-9, CA 50, and CA 72-4 which are associated with stomach cancer; CEA, CA19-9, and Muc-1 which are associated with colon cancer; CA 19-9, Ca-50, and CEA which are associated with pancreas carcinoma; CEA, NSE (neuron specific enolase), and EGF-receptor which are associated with small cell lung cancer; CEA which is associated with lung cancer; α-fetoprotein (AFP) which is associated with liver carcinoma; PSA, PMSA, CDCP1, CD26, Hepsin, HGF (hepatocyte growth factor), Met, CAIX(G250), EphhB4 (Ephrin type-B receptor 4), EGFR1, EGFR2, PDGF, VEGFR, DPP6, syndecan 1, IGFBP2 (Human insulin-like growth factor binding protein 2), CD3, CD28, CTL4, and VEGF which are associated with prostate cancer; CA 19-9 which is associated with gall bladder cancer; SSC (squamous cell carcinoma antigen) which is associated with squamous cell carcinoma; CEA CA 15-3, CEA, BRCA-1, BRCA-2, Muc-1, and Her2/Neu receptor which are associated with mammary carcinoma; AFP and hCG which are associated with testes cancer; CA-125, CEA, CA 15-3, AFP, and TAG-72 which are associated with ovarial carcinoma; and CD20 and CD21 which are associated with B cell lymphoma.

Tumor associated antigens can be identified experimentally or may be selected from a database. Databases that identify molecules that are expressed or upregulated by cancer cells include, for example, the NCI60 microarray project (see e.g., Ross et al., Nature Genetics 24: 227-34 (2000); world wide web at genome-www.stanford.edu/nci60/), the carcinoma classification (see e.g., A. Su et al., Cancer Research 61: 7388-7393 (2001); world wide web at gnf.org/cancer/epican), and the Lymphoma/Leukemia molecular profiling project (see e.g., Alizadeh et al., Nature 403: 503-11 (2000); world wide web at llmpp.nih.gov/lymphoma/). Experimental methods useful for identifying molecules that are expressed or upregulated by cancer cells include, for example, microarray experiments, quantitative PCR, FACS and Northern analysis.

In certain embodiments, the invention provides a composition comprising an APC targeting agent and a free antigen. For example, a composition may comprise an APC targeting agent having an adjuvant and an antigen covalently attached to an APC targeting compound mixed together with antigen which is not attached to the APC targeting compound. In an exemplary embodiment, the antigen attached to the APC targeting compound and the free antigen are the same. The antigen may be a single antigen or a mixture of antigens, including, for example, a variety of peptides from the same protein, peptides from different proteins, or a mixture of other antigen molecules.

In other embodiments, an antigen may be an antigen associated with a pathogen, such as, for example, a plant, bacterium, protozoan, parasite, or virus. Such antigens may be peptides, glycoproteins, or polysaccharides. Specific examples of a wide variety of suitable pathogen antigens may be found, for example, in U.S. Patent Publication No. 2006/0171917.

In certain embodiments, an APC targeting agent is not a two component agent comprising an anti-DC-SIGN antibody complexed to KLH. In certain embodiments, an APC targeting agent is not a mannan coated liposome. In certain embodiments, an APC targeting agent comprises an adjuvant that is not a lipid or liposome. In certain embodiments, an APC targeting agent comprises an antigen that is not a nucleic acid or a nucleic acid vaccine. In certain embodiments, an APC targeting agent comprises an antibody that binds to an APC cell surface molecule, wherein the antibody does not comprise an antigen or an adjuvant incorporated into a complementarity determining region (CDR) or constant region of the antibody.

Also encompassed within the scope of the invention are nucleic acids encoding protein based APC targeting agents, expression vectors comprising nucleic acids encoding the APC targeting agents, and host cells comprising expression vectors for producing the APC targeting agents.

### 3. Methods for Enhancing an Adjuvant Effect

The invention provides methods and compositions for enhancing the effect of an adjuvant by targeting the adjuvant to an antigen presenting cell (APC). The methods utilize an agent comprising an adjuvant linked to a compound that binds to a cell surface protein on an APC. In certain embodiments, the methods may utilize an APC targeting agent comprising an adjuvant and an antigen attached to an APC targeting compound.

The methods may be used to increase the effect of an adjuvant in any situation in which an adjuvant effect is desired, including research purposes as well as therapeutic purposes. For example, adjuvants are commonly used in conjunction with antibody production in order to increase an immune response to a desired antigen administered with the adjuvant. Adjuvants are also commonly used in conjunction with vaccines in order to stimulate an immune response to an antigenic component of the vaccine.

In exemplary embodiments, the invention provides methods for treating a subject in which a stimulation and/or enhancement of an immune response is desirable, e.g., individuals suffering from a pathogen infection, cancer, or other disease state. The methods involve administering to the individual a therapeutically effective amount of one or more APC targeting agents as described herein. These methods are particularly aimed at therapeutic and prophylactic treatments of animals, and more particularly, humans.

In certain embodiments, an APC targeting agent may be administered as part of a combination therapy with one or more other therapeutic agents such as, for example, anti-infective agents (such as for example, antibiotic, antiviral, and/or antifungal compounds, etc.). Exemplary antibiotics include, for example, sulfa drugs (e.g., sulfanilamide), folic acid analogs (e.g., trimethoprim), beta-lactams (e.g., penicillin, cephalosporins), aminoglycosides (e.g., streptomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid). Exemplary antiviral agents include, for example, vidarabine, acyclovir, gancyclovir, valganciclovir, nucleoside-analog reverse transcriptase inhibitors (e.g., AZT, ddI, ddC, D4T, 3TC), non-nucleoside reverse transcriptase inhibitors (e.g., nevirapine, delavirdine), protease inhibitors (e.g., saquinavir, ritonavir, indinavir, nelfinavir), ribavirin, amantadine, rimantadine, relenza, tamiflu, pleconaril, and interferons. Exemplary antifungal drugs include, for example, polyene antifungals (e.g., amphotericin and nystatin), imidazole antifungals (ketoconazole and miconazole), triazole antifungals (e.g., fluconazole and itraconazole), flucytosine, griseofulvin, and terbinafine.

In an exemplary embodiment, the invention provides a method for stimulating an immune response for treating or preventing influenza in a subject, or for treating or ameliorating symptoms associated with influenza. The methods may involve administering a therapeutically effective amount of an APC targeting agent as described herein. Exemplary influenza infections that may be treated in accordance with the methods provided herein include, for example, influenza types A, B and C. In an exemplary embodiment, the influenza is influenza A, such as, for example: A/PR/8/34 or A/HK/8/68, or selected from H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3. In various embodiments, an APC targeting agent may be administered substantially contemporaneously with or following infection of the subject, i.e., a therapeutic treatment. In other embodiments, the APC targeting agent provides a therapeutic benefit, such as, reducing or decreasing one or more symptoms or complications of influenza infection, virus titer, virus replication or an amount of a viral protein of one or more influenza strains. Symptoms or complications of influenza infection that can be reduced or decreased include, for example, chills, fever, cough, sore throat, nasal congestion, sinus congestion, nasal infection, sinus infection, body ache, headache, fatigue, pneumonia, bronchitis, ear infection, earache or death. In still another embodiment, a therapeutic benefit includes hastening a subject's recovery from influenza infection. In still other embodiments, an APC targeting agent may be administered as part of a combination therapy with an anti-viral agent or one or more agents that inhibit one or more symptoms or complications associated with influenza infection (e.g., chills, fever, cough, sore throat, nasal congestion, body ache, headache, fatigue, pneumonia, bronchitis, sinus infection or ear infection). In one embodiment, the APC targeting agent useful for treating or preventing influenza comprises an adjuvant covalently linked to an APC targeting compound. In another embodiment, an APC targeting agent useful for treating influenza comprises an APC targeting compound having an adjuvant and an influenza antigen covalently attached thereto.

In certain embodiments, methods of inhibiting or reducing tumor growth and methods of treating an individual suffering from cancer are provided. The methods involve administering to the individual a therapeutically effective amount of one or more APC targeting agents as described herein. In certain embodiments, the APC targeting agent may comprise an adjuvant covalently attached to an APC targeting compound. In other embodiments, an APC targeting agent may comprise an APC targeting complex with an adjuvant and a tumor associated antigen covalently attached thereto. These methods are particularly aimed at therapeutic and prophylactic treatments of animals, and more particularly, humans.

APC targeting agents may be useful for treating or preventing a cancer (tumor), including, but not limited to, colon carcinoma, breast cancer, mesothelioma, prostate cancer, bladder cancer, squamous cell carcinoma of the head and neck (HNSCC), Kaposi's sarcoma, glioblastoma, astrocytoma, lymphoma, lung carcinoma, melanoma, renal cancer, and leukemia.

In certain embodiments, one or more APC targeting agents can be administered together (simultaneously) or at different times (sequentially). The APC targeting agents can be used alone or in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of one or more APC targeting agents of the invention.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), troponin subunits, antagonists of vitronectin αᵥβ₃, peptides derived from Saposin B, antibiotics or analogs (e.g., tetracycline or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of an APC targeting agent of the invention may be continued while the other therapy is being administered and/or thereafter. Administration of the APC targeting agent may be made in a single dose, or in multiple doses. In some instances, administration of the APC targeting agent is commenced at least several days prior to the conventional therapy, while in other instances, administration is begun either immediately before or at the time of the administration of the conventional therapy.

In exemplary embodiments, the methods described herein involve enhancing an adjuvant effect or stimulating an immune response in a subject which has not previously been vaccinated with the adjuvant which is attached to the APC targeting compound.

In certain embodiments, the methods described herein may stimulate a naive immune response, a recall immune response, or both.

In exemplary embodiments, the methods described herein stimulate an immune response to an antigen other than the adjuvant or the APC targeting compound. In certain embodiments, the methods may involve stimulating an immune response to an unknown antigen. In certain embodiments, the methods may involve stimulating an immune response to an antigen already present in the subject, such as, for example, a tumor antigen or a pathogen antigen.

In certain embodiments, when utilizing a three component APC targeting agent, e.g., comprising an antigen, adjuvant and APC targeting compound, the methods involve stimulating an immune response to the associated antigen rather than to the adjuvant or the APC targeting compound (although this does not exclude also stimulating an immune response to the adjuvant and/or APC targeting compound). The subject may, or may not, have been previously exposed or vaccinated with said antigen.

### 4. Antibody Production

Antibodies useful for production of certain of the APC targeting agents described herein may be designed to bind to a desired epitope or may be selected from publicly available sources of known antibodies. For example, databases of antibody sequences may be found on the world wide web at imgt.cines.fr. Nucleic acid sequences encoding an antibody may be manipulated to add one or more sequences, such as a protein adjuvant or peptide antigen, using standard recombinant DNA techniques. The nucleic acid sequences encoding the protein based APC targeting agents may be introduced into an expression vector and a suitable host cell for expression of the APC targeting agents as described further below.

Preparation of immunizing antigen, and polyclonal and monoclonal antibody production can be performed as described herein, or using other suitable techniques. A variety of methods have been described. See e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991). Generally, a hybridoma can be produced by fusing a suitable immortal cell line (e.g., a myeloma cell line such as SP2/0) with antibody producing cells. The antibody producing cells, preferably those of the spleen or lymph nodes, are obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807.

To illustrate, immunogens derived from an APC cell surface protein can be used to immunize a mammal, such as a mouse, a hamster or rabbit. See, for example, Antibodies: A Laboratory Manual ed. by Harlow and Lane (Cold Spring Harbor Press: 1988). Techniques for conferring immunogenicity on a protein or peptide include conjugation to carriers or other techniques well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a desired polypeptide and monoclonal antibodies isolated from a culture comprising such hybridoma cells.

In certain embodiments, antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. Fab fragments can be generated by treating antibody with papain.

In certain embodiments, antibodies described herein are further intended to include bispecific, single-chain, and chimeric and humanized molecules having affinity for a polypeptide of interest conferred by at least one CDR region of the antibody. Techniques for the production of a light chain or heavy chain dimers, or any minimal fragment thereof such as an Fv or a single chain (scFv) construct are described, for example, in U.S. Pat. No. 4,946,778. Also, transgenic mice or other organisms including other mammals may be used to express humanized antibodies. Methods of generating these antibodies are known in the art. See, e.g., Cabilly et al., U.S. Pat. No. 4,816,567; Cabilly et al., European Patent No. 0,125,023; Queen et al., European Patent No. 0,451,216; Boss et al., U.S. Pat. No. 4,816,397; Boss et al., European Patent No. 0,120,694; Neuberger, M. S. et al., WO 86/01533; Neuberger, M. S. et al., European Patent No. 0,194,276; Winter, U.S. Pat. No. 5,225,539; Winter, European Patent No. 0,239,400; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)).

Such humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

A method for generating a monoclonal antibody that binds specifically to a desired polypeptide may comprise administering to a mouse an amount of an immunogenic composition comprising the polypeptide in an amount effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the polypeptide. Once obtained, a hybridoma can be propagated in a cell culture, optionally in culture conditions where the hybridoma-derived cells produce the monoclonal antibody that binds specifically to polypeptide. The monoclonal antibody may be purified from the cell culture.

In addition, the hybridoma cell lines can be used as a source of nucleic acids encoding the immunoglobulin chains, which can be isolated and expressed (e.g., upon transfer to other cells using any suitable technique (see e.g., Cabilly et al., U.S. Pat. No. 4,816,567; Winter, U.S. Pat. No. 5,225,539)). For instance, clones comprising a rearranged light or heavy chain can be isolated (e.g., by PCR) or cDNA libraries can be prepared from mRNA isolated from the cell lines, and cDNA clones encoding a desired immunoglobulin chain can be isolated. Thus, nucleic acids encoding the heavy and/or light chains of the antibodies, or portions thereof, can be obtained and used in accordance with recombinant DNA techniques for the production of the specific immunoglobulin, immunoglobulin chain, or variants thereof (e.g., humanized immunoglobulins) in a variety of host cells or in an in vitro translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host cell by an appropriate method (e.g., transformation, transfection, electroporation, infection), such that the nucleic acid is operably linked to one or more expression control elements (e.g., in the vector or integrated into the host cell genome). For production, host cells can be maintained under conditions suitable for expression (e.g., in the presence of inducer, suitable media supplemented with appropriate salts, growth factors, antibiotic, nutritional supplements, etc.), whereby the encoded polypeptide is produced. If desired, the encoded protein can be recovered and/or isolated (e.g., from the host cells or medium). It will be appreciated that the method of production encompasses expression in a host cell of a transgenic animal (see e.g., WO 92/03918, GenPharm International, published Mar. 19, 1992).

Antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage, including fd and M13. The antigen binding domains are expressed as a recombinantly fused protein to either the phage gene III or gene VIII protein. Examples of phage display methods that can be used to make the immunoglobulins, or fragments thereof, of the present invention include those disclosed in Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired fragments, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques, 12(6):864-869, 1992; and Sawai et al., Am. J. Reprod. Immunol., 34:26-34, 1995; and Better et al., Science, 240:1041-1043, 1988 (each of which is incorporated by reference in its entirety). Examples of techniques which can be used to produce single-chain Fvs and antibodies include those described in U.S. Pat. Nos. 4,946,778 and 5,258,498; Huston et al., Methods in Enzymology, 203:46-88, 1991; Shu et al., Proc. Natl. Acad. Sci. USA, 90:7995-7999, 1993; and Skerra et al., Science, 240:1038-1040, 1988.

Polynucleotides encoding antibodies having a desired binding specificity may be obtained by any method known in the art. The nucleotide sequence of antibodies immunospecific for a desired antigen can be obtained, for example, as described above, from the literature or from a database such as GenBank. Polynucleotides encoding an antibody may be assembled from chemically synthesized oligonucleotides (e.g., as described in Kutmeier et al., BioTechniques 17: 242 (1994)), which, briefly, involves the synthesis of overlapping oligonucleotides containing portions of the sequence encoding the antibody, annealing and ligating of those oligonucleotides, and then amplification of the ligated oligonucleotides by PCR. Alternatively, a polynucleotide encoding an antibody may be produced from a cDNA library obtained from a tissue or cell expressing the antibody such as a hybridoma cell line selected to express an antibody. The desired antibody genes may be isolated from the library by PCR amplification using synthetic primers hybridizable to the 3′ and 5′ ends of the sequence. Amplified nucleic acids generated by PCR may then be cloned into replicable cloning vectors using any method well known in the art.

Once a nucleic acid sequence encoding an APC targeting agent has been obtained, the vector for the production of the APC targeting agent may be produced by recombinant DNA technology using techniques well known in the art. Methods which are well known to those skilled in the art can be used to construct expression vectors containing the APC targeting agent coding sequences and appropriate transcriptional and translational control signals. These methods include, for example, in vitro recombinant DNA techniques, synthetic techniques, and in vivo genetic recombination. (See, for example, the techniques described in Sambrook et al., 1990, Molecular Cloning A Laboratory Manual, 2d Ed., Cold Spring Harbor Laboratory, Cold Spring Harbor, N.Y. and Ausubel et al. eds., 1998, Current Protocols in Molecular Biology, John Wiley & Sons, NY).

An expression vector comprising the nucleotide sequence of an APC targeting agent (or a component thereof, such as an antibody, protein adjuvant, antigen peptide, etc.) can be transferred to a host cell by conventional techniques (e.g., electroporation, liposomal transfection, and calcium phosphate precipitation) and the transfected cells are then cultured by conventional techniques to produce the antibody. In specific embodiments, the expression of the APC targeting agent is regulated by a constitutive promoter, an inducible promoter, or a tissue specific promoter.

The host cells used to express the recombinant APC targeting agents may be either bacterial cells (such as Escherichia coli) or eukaryotic cells. Eukaryotic cells may be particularly useful for the expression of APC targeting agents comprising a whole recombinant immunoglobulin molecule. For example, mammalian cells such as Chinese hamster ovary cells (CHO), in conjunction with a vector such as the major intermediate early gene promoter element from human cytomegalovirus is an effective expression system for immunoglobulins (Foecking et al., 1998, Gene 45:101; Cockett et al., 1990, Bio/Technology 8:2).

A variety of host-expression vector systems may be utilized to express the APC targeting agents described herein. Such host-expression systems represent vehicles by which the coding sequences of the APC targeting agents may be produced and subsequently purified, but also represent cells which may, when transformed or transfected with the appropriate nucleotide coding sequences, express the APC targeting agents in situ. These include, but are not limited to, microorganisms such as bacteria (e.g., E. coli and B. subtilis) transformed with recombinant bacteriophage DNA, plasmid DNA or cosmid DNA expression vectors containing immunoglobulin coding sequences; yeast (e.g., Saccharomyces or Pichia) transformed with recombinant yeast expression vectors containing immunoglobulin coding sequences; insect cell systems infected with recombinant virus expression vectors (e.g., baculovirus) containing the immunoglobulin coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing immunoglobulin coding sequences; or mammalian cell systems (e.g., COS, CHO, BHK, 293, 293T, 3T3 cells, lymphatic cells (see U.S. Pat. No. 5,807,715), Per C.6 cells (rat retinal cells developed by Crucell)) harboring recombinant expression constructs containing promoters derived from the genome of mammalian cells (e.g., metallothionein promoter) or from mammalian viruses (e.g., the adenovirus late promoter; the vaccinia virus 7.5K promoter). Methods for making antibodies in plants, yeast or fungi/algae that are applicable to the production of the APC targeting agents described herein are described, for example, in U.S. Pat. Nos. 6,046,037 and 5,959,177 and U.S. Patent Publication Nos. 2005/0037420 and 2005/0138692.

In bacterial systems, a number of expression vectors may be advantageously selected depending upon the use intended for the APC targeting agent being expressed. For example, when a large quantity of such a protein is to be produced, for the generation of pharmaceutical compositions of an APC targeting agent, vectors which direct the expression of high levels of fusion protein products that are readily purified may be desirable. Such vectors include, but are not limited to, the E. coli expression vector pUR278 (Ruther et al., 1983, EMBO J. 2:1791), in which the APC targeting agent coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such APC targeting agents are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The APC targeting agent coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the APC targeting agent coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (see e.g., Logan & Shenk, Proc. Natl. Acad. Sci. USA 81: 355-359 (1984)). Specific initiation signals may also be required for efficient translation of inserted APC targeting agent coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153: 51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, W138, BT483, Hs578T, HTB2, BT20 and T47D, CRL7030 and Hs578Bst.

For long-term, high-yield production of recombinant proteins (e.g., APC targeting agents, antibodies, protein adjuvants, peptide antigens, etc.), stable expression is preferred. For example, cell lines which stably express an APC targeting agent may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the APC targeting agents described herein.

A number of selection systems may be used, including but not limited to the herpes simplex virus thymidine kinase (Wigler et al., Cell 11: 223 (1977)), hypoxanthine-guanine phosphoribosyltransferase (Szybalska & Szybalski, Proc. Natl. Acad. Sci. USA 48: 202 (1992)), and adenine phosphoribosyltransferase (Lowy et al., Cell 22: 817 (1980)) genes can be employed in tk−, hgprt− or aprt-cells, respectively. Also, antimetabolite resistance can be used as the basis of selection for the following genes: dhfr, which confers resistance to methotrexate (Wigler et al., Proc. Natl. Acad. Sci. USA 77: 357 (1980); O'Hare et al., Proc. Natl. Acad. Sci. USA 78: 1527 (1981)); gpt, which confers resistance to mycophenolic acid (Mulligan & Berg, Proc. Natl. Acad. Sci. USA 78: 2072 (1981)); neo, which confers resistance to the aminoglycoside G-418 (Tolstoshev, Ann. Rev. Pharmacol. Toxicol. 32: 573-596 (1993); Mulligan, Science 260: 926-932 (1993); and Morgan and Anderson, Ann. Rev. Biochem. 62: 191-217 (1993); May, TIB TECH 11: 155-215 (1993)); and hygro, which confers resistance to hygromycin (Santerre et al., Gene 30: 147 (1984)). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1.

The expression levels of an APC targeting agent (or a component thereof) described herein can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an APC targeting agent is amplifiable, increase in the level of inhibitor present in culture of host cell will increase the number of copies of the marker gene. Since the amplified region is associated with the nucleotide sequence of the APC targeting agent, production of the APC targeting agent will also increase (Crouse et al., 1983, Mol. Cell. Biol. 3:257).

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once an APC targeting agent (or a component thereof) of the invention has been recombinantly expressed, it may be purified by any method known in the art for purification of an antibody, for example, by chromatography (e.g., ion exchange, affinity, particularly by affinity for the specific antigen after Protein A, and sizing column chromatography), centrifugation, differential solubility, or by any other standard technique for the purification of proteins.

### 5. Pharmaceutical Compositions

The invention provides methods and pharmaceutical compositions comprising one or more APC targeting agents. The invention also provides methods of treatment, prophylaxis, and amelioration of one or more symptoms associated with a disease, disorder or infection by administering to a subject an effective amount of an APC targeting agent, or a pharmaceutical composition comprising an APC targeting agent. In one embodiment, an APC targeting agent is substantially purified (i.e., substantially free from substances that limit its effect or produce undesired side-effects). Subjects that may be treated with an APC targeting agent described herein include, for example, an animal, such as a mammal including non-primates (e.g. cows, pigs, horses, cats, dogs, rats, etc.) and primates (e.g., monkey, such as a cynomolgous monkey, and a human). In an exemplary embodiment, the subject is a human.

In certain embodiments, the APC targeting agents described may be formulated with a pharmaceutically acceptable carrier. Such APC targeting agents can be administered alone or as a component of a pharmaceutical formulation (composition). The APC targeting agents may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject APC targeting agents include those suitable for oral, dietary, topical, parenteral (e.g., intravenous, intraarterial, intramuscular, subcutaneous injection), inhalation (e.g., intrabronchial, intranasal or oral inhalation, intranasal drops), rectal, and/or intravaginal administration. Other suitable methods of administration can also include rechargeable or biodegradable devices and slow release polymeric devices. The pharmaceutical compositions described herein can also be administered as part of a combinatorial therapy with other therapeutics (either in the same formulation or in a separate formulation).

The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the APC targeting agent which produces a therapeutic effect.

In certain embodiments, methods of preparing these formulations or compositions include combining an APC targeting agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more subject APC targeting agents as an active ingredient.

In solid dosage forms for oral administration (capsules, tablets, pills, dragees, powders, granules, and the like), one or more APC targeting agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Methods of the invention can utilize topical administration, either to skin or to mucosal membranes such as those on the cervix and vagina. This may offer direct delivery to a tumor while avoiding the induction of side effects. Topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject APC targeting agents may be mixed under sterile conditions with a pharmaceutically acceptable carrier, and with any preservatives, buffers, or propellants which may be required. The ointments, pastes, creams and gels may contain, in addition to an APC targeting agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more APC targeting agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsuled matrices of one or more APC targeting agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal or rectal administration may be presented as a suppository, which may be prepared by mixing one or more agents of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### In Vivo Targeting of an Adjuvant to Dendritic Cells Through DC-SIGN

DCs are antigen-presenting cells with the unique ability to take up and process antigens in the peripheral blood and tissues and present them to the lymphocytes in the draining lymph nodes for the activation of an immune response. However, for a productive T cell response to occur, an activation stimulus is required, otherwise, tolerance will be induced (Hawiger, D., et al. 2001. J Exp Med 194:769-779). Development of cancer vaccines has focused primarily on ex vivo loading of autologous DCs (Banchereau, J., et al. 2005. Nat Rev Immunol 5:296-306). Recently, a more direct approach of targeting antigen in vivo to antigen uptake receptors on DCs has been explored in pre-clinical studies. Potential candidates of interest are Fc receptors (Regnault, A., et al. 1999. J Exp Med 189:371-380; Kalergis, A. M., et al. 2002. J Exp Med 195:1653-1659), chemokine receptors (Biragyn, A., et al. 2004. Blood 104:1961-1969) and C-type lectins (Bonifaz, L., et al. 2002. J Exp Med 196:1627-1638; Ramakrishna, V., et al. 2004. J Immunol 172:2845-2852). While Fc and chemokine receptor expression is not limited to DCs, some members of the C-type lectin family show a more DC specific expression pattern. The type I C-type lectins, e.g., mannose receptor and DEC-205, have been used for in vitro and in vivo antibody targeting studies. Antibodies to both receptors can specifically deliver antigen to DCs, which is appropriately processed and presented to T-cells. However, the observed T cell response turned out to be tolerogenic (Bonifaz, L., et al. 2002. J Exp Med 196:1627-1638; Chieppa, M., et al. 2003. J Immunol 171:4552-4560). An additional co-stimulatory signal was required to elicit a sufficient stimulatory response when antigens are delivered via DEC 205 antibodies.

In contrast, DC-SIGN is a type II C-type lectin with an expression pattern distinct from DEC205 on human DC subsets. DC-SIGN is also an endocytic receptor capable of efficiently taking up antigens as demonstrated by its ability to directly bind envelope proteins on a variety of pathogenic viruses (Simmons, G., et al. 2003. Virology 305:115-123; Geijtenbeek, T. B., et al. 2000. Cell 100:587-597; Cambi, A., et al. 2005. Cell Microbiol 7:481-488). In vitro studies demonstrated that a DC-SIGN antibody-KLH conjugate was internalized by DCs, and KLH epitopes were successfully presented on the surface of the DCs, inducing an anti-KLH T cell response (Tacken, P. J., et al. 2005. Blood 106:1278-1285). The study presented herein evaluates whether targeting of antigen through DC-SIGN in vivo raises a stimulatory immune response of naïve human immune cells. In humans, DC-SIGN is only expressed on professional antigen presenting cells, whereas in mice, DC-SIGN biology is much more complex with multiple family members and different expression patterns (Park, C. G., et al. 2001. Int Immunol 13:1283-1290; Takahara, K., et al. 2004. Int Immunol 16:819-829). Therefore, antibody-mediated targeting of DC-SIGN in a strictly murine model might not translate to the human setting. This study used immunodeficient Rag2^{-/-}γc^{-/-} mice reconstituted with the essential cells of the human immune system (Traggiai, E., et al. 2004. Science 304:104-107) as a model to evaluate in vivo targeting of antigens to DCs using DC-SIGN antibodies. The anti-DC-SIGN antibodies hD1V1 and E10 do not cross-react with any murine DC-SIGN family members, therefore targeting in the reconstituted Rag2^{-/-}γc^{-/-} is entirely through human DC-SIGN. Keyhole limpet hemocyanin (KLH) and tetanus toxoid (TT) were used as model antigens, and were either chemically or genetically linked to anti-DC-SIGN antibodies. Targeted delivery of either antigen to DCs resulted in a proliferative human immune response in the absence of any additional co-stimulatory signals. Furthermore, a tumor xenograft model reconstituted with human peripheral blood lymphocytes (hPBL) was used to evaluate whether administration of KLH linked to an anti-DC-SIGN antibody can enhance the adjuvant effect observed with KLH in other tumor models and human clinical trials (Ragupathi, G., et al. 2003. Clin Cancer Res 9:5214-5220; Krug, L. M., et al. 2004. Clin Cancer Res 10:6094-6100; Slovin, S. F. 2005. Clin Prostate Cancer 4:118-123). Tumor growth inhibition was significantly enhanced in a xenograft tumor model by KLH linked to a DC-SIGN antibody. Therefore, targeting antigens to DCs via DC-SIGN antibodies could be a powerful method to raise strong and long-lasting antigen-specific T cell responses against cancer and infectious diseases.

### Materials and Methods

Antibody constructs. KLH was conjugated to the humanized anti-DC-SIGN antibody hD1V1 specific for human DC-SIGN containing a G2G4 fusion constant region by chemical conjugation using sulfosuccinimidyl 4-(N-maleimidomethyl)cyclohexane-1-carboxylate (sulfo-SMCC; Pierce, Rockford, Ill.) as described in Tacken et al. (Blood 106:1278-1285 (2005)). Similarly, KLH was also conjugated to the control antibody 5G1.1 (a humanized IgG2/G4 antibody recognizing C5a).

scFv-RR-TT is a single chain variant of hD1V1. scFvD1V1 was first cloned into APEX3P, followed by the attachment of two copies of a universal TT helper epitope amino acids 829-844 at the C-termini in tandem with arginines ‘RR’ linkers for proteasomal cleavage. D1V1 Vₖ leader and Vₖ sequences, and Vₕ of the humanized anti-DC-SIGN D1V1 genes were amplified. A 6×His for purification, stop codon and XbaI/EcoRI enzyme cloning sites were introduced. After overlap PCR, the sequence encoding a (Gly-Gly-Gly-Gly-Ser)₃ (SEQ ID NO: 6) linker was introduced between Vₖ and Vₕ. The PCR fragment was TA cloned into pCR2.1 vector and the correct sequence was confirmed. The pCR2.1-scFv-D1V1 was then digested with BamHI/XbaI and the fragment was cloned into Alexion's mammalian cell expression vector Apex3P (Apex3P-scFv D1V1HIS). The final construct was confirmed by sequencing.

293 EBNA human embryonic kidney cells transfected with Apex3P-scFvD1V1-RR-TT by Effectene (Qiagen) were grown in DMEM (Cellgro #10-013-CV) with 10% heat inactivated FBS, 100 IU/mL penicillin, 100 μg/mL streptomycin, 2 mM glutamine with 250 active μg/mL G418 Sulfate and 1 μg/mL puromycin at 37° C. and 5% CO₂. 90-95% confluent T-175 flasks of cells were washed with 15 mL Hank's Balanced Salt Solution (HBSS) (or PBS with Ca/Mg) in order to remove serum proteins before the addition of 30 mL IS Pro serum free medium (Irvine Scientific, Santa Ana, Calif., Catalog #91103) supplemented with L-Glutamine and penicillin/streptomycin to each flask. The supernatant was concentrated and purified by a Qiagen Ni-NTA Column.

Human DC-SIGN/L-SIGN cross-reactive chimeric antibody E10-TT (Isotype, IgG1) embedded with TT epitope was constructed as previously described (Dakappagari, N., et al. 2006. J Immunol 176:426-440) by inserting TT epitope 630DR by overlap PCR into the CH2 domain between glycines 249 and 250 with two additional arginines upstream and three downstream of the epitope to give a final insertion of 25 total amino acids (RRIDKISDVSTIVPYIGPALNIRRR) (SEQ ID NO: 7). All antibodies used in the studies tested negative for endotoxin as determined by using the QCL-1000 Limulus amebocyte lysate assay (BioWhittaker, Walkersville, Md.). A functional grade anti-CD40 antibody was purchased from eBioscience (San Diego, Calif.).

Cord blood samples and progenitor cell isolation. Fresh umbilical cord blood was obtained from Advanced Bioscience Resources, Inc. (Alameda, Calif.) after parental informed consent. The blood tested negative for HIV, Hepatitis B surface antigen, Hepatitis C and Cytomegalo Virus. Cord blood mononuclear cells were isolated by Ficoll-Paque gradient centrifugation (Histopaque, Sigma-Aldrich). Then blood progenitor cells were isolated using a Progenitor enrichment kit from Stem Cell Technologies, Inc. (Vancouver, Calif.). The progenitor cells were aliquoted and stored in liquid nitrogen until use.

Mice. Breeding pairs of Rag2^{-/-}γc^{-/-} mice on a BALB/c background were purchased from the Central Institute for Experimental Animals, Kanagawa, Japan. The mice were bred and housed in specific pathogen-free conditions at Perry Scientific (San Diego, Calif.). Four to six week old NOD.CB17-Prkdcscid/J (NOD/SCID) mice were obtained from Jackson Laboratory (Bar Harbor, Me.) and maintained under specific pathogen-free conditions. All work with animals was approved by Perry Scientific's Institutional Animal Care and Use Committee (IACUC).

Engraftment of newborn Rag2^{-/-}γc^{-/-} mice. Newborn mice were engrafted with cord blood cells based on the method described by Traggiai et al (Science 304:104-107 (2004)) with some modifications. All procedures were approved by Perry Scientific's IACUC committee. Newborn mice were irradiated with 1Gy at 1Gy/min (University of California, San Diego). At 1-2 hours post irradiation, mice were injected with CD34+ cord blood cells in 30 μL RPMI into the liver using a 30 gauge ½ inch needle (Becton Dickinson and Company, Franklin Lakes, N.J.). Cord blood cell grafted pups were weaned at 3-4 weeks of age.

Cell isolation. Peripheral blood was drawn from each mouse via retro orbital puncture. Peripheral blood mononuclear cells (PBMC) were separated from heparinized whole blood by density gradient centrifugation. Femurs and tibias were removed aseptically. Bone marrow was flushed from either end with complete RPMI-1640 (Invitrogen). The cell suspension was filtered through a cell strainer (Falcon; BD Biosciences) and centrifuged at 400×g for 8 minutes to pellet cells. Single cell suspension from spleen, lymph nodes (superficial cervical, axillary, inguinal, and mesenteric) were prepared using standard procedures. The red blood cells in spleen, PBMC, and bone marrow were lysed by ammonium chloride (Stem Cell Technologies Inc., Vancouver, BC). The cells were washed twice with RPMI-1640 before using in further experiments.

Immunization of cord-blood engrafted mice. Mice received KLH (Calbiochem (EMD Biosciences), San Diego, Calif.) or TT antigens (Synpep, Dublin, Calif.) in Titermax adjuvant either s.c. or i.p. while DC-SIGN antibodies D1V1-KLH, 5G1.1-KLH or D1V1-KLH, and CD40 antibodies were administered intravenously in 200 μL PBS.

Proliferation assays. Spleens were removed, and single-cell suspensions were prepared aseptically. After centrifugation, red blood cells were lysed by ammonium chloride (Stem Cell Technologies Inc., Vancouver, BC). 2×10⁵ spleen cells were cultured in 96-well plates with KLH or TT in RPMI 1640 supplemented with 10% FCS, 10 mM Hepes, 1 mM Sodium Pyruvate Solution, 2-mM L-glutamine, 0.075% (w/v) sodium bicarbonate, and antibiotics for 5 days. After 4 days of co-culture, 3H-thymidine (1 μCi/well; Amersham, Piscataway, N.J.) was added to the cell cultures and thymidine incorporation was measured after 16-18 hours on a microplate scintillation counter (Perkin Elmer, Shelton, Conn.). Proliferation index was determined as (cpm of KLH or TT treatment)/(cpm of medium treatment).

Flow Cytometry analysis. Immune cells isolated from engrafted mice were resuspended in PBS with 1% BSA and 0.1% NaN₃, and incubated for 20 minutes with one of the following: PE-conjugated anti-human CD19, APC-conjugated anti-human CD4, PE-conjugated anti-human CD8, or FITC-conjugated anti-human CD209 (eBiosciences, San Diego, Calif.) and washed twice in the same buffer. The cells were analyzed on a FACSCalibur flow cytometer and data analyzed using BD CellQuest Pro software (BD Biosciences, Mountain View, Calif.). The experimental data were expressed as the percentage of gated cells stained positive for the given marker.

RAJI-PBL model. Peripheral blood was drawn from healthy human donors at the San Diego Blood Bank after informed consent and IRB approval. PBMCs were isolated by centrifugation of the blood over a Histopaque gradient (Sigma) at 400×g for 15 minutes. Cells were washed twice in RPMI-1640 medium. Immature DCs were prepared by isolating CD14+ monocytes using magnetic separation with monocyte isolation kit II (Miltenyi Biotec) from the same donor. The cells were then cultured for 5 days in the presence of 800 U/mL human recombinant GM-CSF (Stemcell Technologies), 500 U/mL human recombinant IL-4 (Stemcell Technologies). GM-CSF and IL-4 were added again on Day 3.

RAJI cells (ATCC) were cultured in RPMI supplemented with 10% FCS. For fludarabine (Sigma) treated RAJI cells, RAJI cells were incubated with fludarabine at 5 μg/mL for 24 h, washed three times in RPMI-1640 medium. Four million RAJI cells, 30,000 immature DCs, and 3 million human PBMC were injected s.c. into NOD/SCID mice. Tumor growth was determined by microcaliper measurements by personnel blinded to the study design. Tumor volume was calculated according to the formula (length)×(width)×(width)/2. The experimental data represent mean of tumor volumes from ten mice of each group. Antibody treatment was initiated the day of tumor and PBMC injection by s.c. injection at 1/10 of the indicated dose, followed by weekly s.c. injections in tumor proximity over 2 weeks.

Statistical Analysis. Differences between groups were analyzed by two-tailed unpaired Student's t test. Significance was accepted when p<0.05.

### Results

In vivo targeting of KLH by DC-SIGN antibody hD1V1 in cord blood engrafted mice induces a stimulatory immune response. Tacken et al. (Blood 106:1278-1285 (2005)) demonstrated that in vitro incubation of DCs with a humanized anti-DC-SIGN antibody (hD1V1-G2G4) conjugated to KLH results in internalization of the construct, and successful presentation of KLH epitopes to T cells from a patient immunized with KLH. The resulting proliferative T cell response was 100-fold stronger than observed by stimulation with unconjugated KLH. The present study addresses whether D1V1-KLH could successfully target DCs in vivo and stimulate naïve human immune cell responses. Since the hD1V1-G2G4 antibody does not cross-react with rodent DC-SIGN, and the DC-SIGN biology in mice appears to be substantially different from humans (Park, C. G., et al. 2001. Int Immunol 13:1283-1290; Takahara, K., et al. 2004. Int Immunol 16:819-829), mice engrafted with human immune cells appeared the best choice to evaluate in vivo targeting of D1V1-KLH.

The human cord blood engrafted mice described by Traggiai et al. (Science 304:104-107 (2004)) have been reported to develop a complete range of functional human immune cells. To confirm that transplantation of human hematopoietic stem and progenitor cells into the liver of immunodeficient newborn mice results in engraftment, expansion, and overall reconstitution of a human immune system, human DCs, human B cells, human CD8 and CD4 T cells from various immune organs in a number of the grafted mice were analyzed by flow cytometry. At three months after cell injection, there were about 12%-25% of CD19 positive cells, 20-31% of CD8+ cells, and 11-15% of CD4+ cells in spleen and lymph node (Table I). The percentages of B cells and DCs appear comparable to those in humans, but the CD4/CD8 ratio was reversed compared to what is expected in humans. These data imply that human stem cells and progenitor cells in the reconstituted Rag2^{-/-}γc^{-/-} mice develop into T and B cells over three months as previously reported (Traggiai, E., et al. 2004. Science 304:104-107).

TABLE I
Cell frequency (%) after cord blood engraftment. Three months after
injection of human cord blood cells into Rag2^{-/-}γc^{-/-} mice,
bone marrow, lymph nodes, spleens were harvested and
cell phenotype was assessed by flow cytometry
using antibodies to human CD antigens

| | DC | B Cell | | T Cell | |
|---|---|---|---|---|---|
| | CD11c/CD209 | CD19 | CD20 | CDS | CD4 |
| Mouse #1 | | | | | |
| Blood | 1.23 | 19.84 | 8.74 | 12.19 | 13.31 |
| Bone Marrow | 1.8 | 4.87 | 16.54 | 30.56 | 13.82 |
| Lymph Node | 0.53 | 24.41 | 3.91 | 20.51 | 13.88 |
| Spleen | 3.04 | 9.98 | 15.77 | 22.1 | 13.77 |
| Mouse #2 | | | | | |
| Blood | 0.63 | 8.98 | 17.76 | 13.19 | 12.82 |
| Bone Marrow | 2.48 | 22.98 | 18.03 | 19.37 | 14.05 |
| Lymph Node | 0.43 | 22.54 | 2.29 | 19.75 | 10.79 |
| Spleen | 3.6 | 15.14 | 25 | 21.51 | 14.94 |

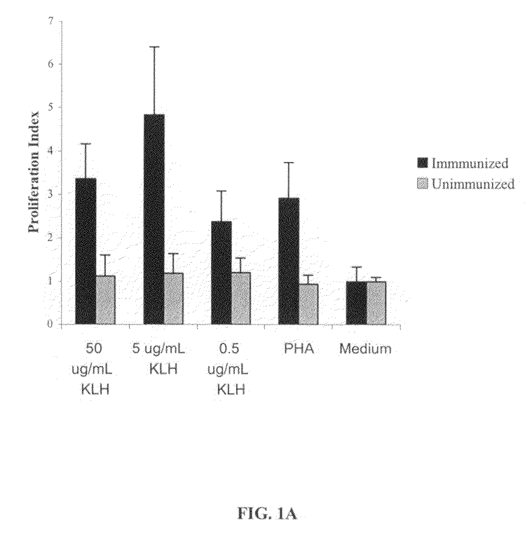

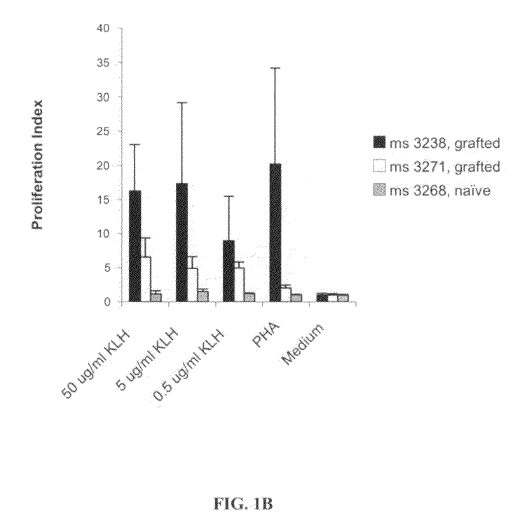

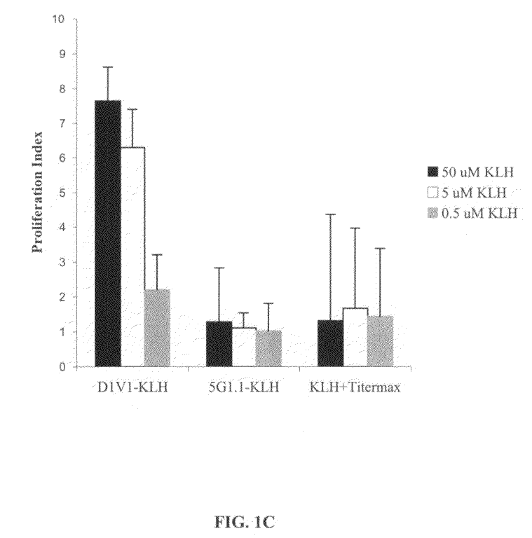

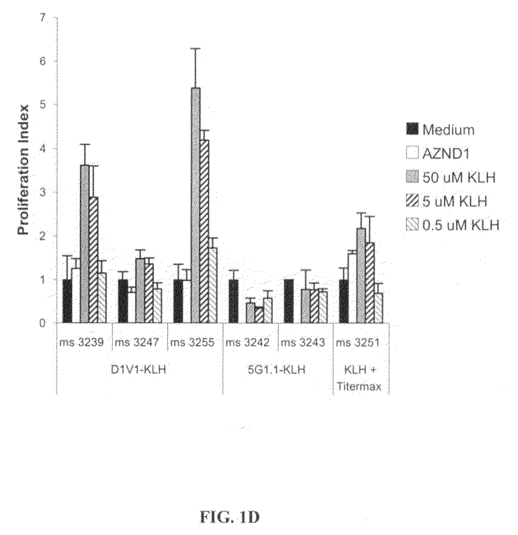

Before testing targeted delivery of KLH to DCs, the human T cells in the engrafted mice were evaluated for the ability to mount a detectable proliferative immune response to KLH following immunization in an adjuvant. Splenocytes isolated from KLH vaccinated mice 9 days after vaccination proliferated when stimulated with KLH in vitro, in contrast to splenocytes from unvaccinated mice (FIG. 1A). A second immunization with KLH did not further increase the proliferative response (FIG. 1B). Overall, the proliferation indices were fairly low, presumably reflecting fairly low human T cell numbers. However, the responses are consistent and reproducible, indicating that the human cord blood grafted mice are a suitable model for testing targeted delivery of KLH through DC-SIGN.

To test specific targeting of antigen to DCs in vivo, Rag2^{-/-}γc^{-/-} grafted mice were immunized with either D1V1-KLH or KLH conjugated to a control antibody recognizing the complement protein C5a (5G1.1-KLH) to ensure any response to the KLH antibody conjugates is mediated through DC-SIGN. Spleens were isolated after 9 days, and the proliferative response to KLH was assessed in vitro. Mice that received D1V1-KLH showed a strong proliferative response to KLH of about 7-fold at 5 μM and 6-fold at 50 μM, while mice that received 5G1.1-KLH control antibody did not show a strong proliferative response (FIG. 1C). Even 14 days after immunization, two of three mice immunized with D1V1-KLH showed good T cell proliferation in response to KLH, of two to four fold compared to control antibody 5G1.1-KLH at 5 μM KLH and three to five fold at 50 μM KLH (FIG. 1D). The responses to KLH induced by D1V1-KLH immunizations were comparable to proliferation induced by PHA, indicating that a strong response to KLH was provoked. None of the two 5G1.1-KLH immunized mice showed a T cell response to KLH, indicating that targeting through DC-SIGN is critical to raise an immune response against KLH. In contrast, responses in mice immunized with KLH in Titermax were weak, but significant.

On detailed analysis, it was found that human immune cells were detectable in all grafted mice. The numbers varied up to 2-fold among mice, but higher numbers of human cells did not correlate with stronger immune responses to KLH (Table II). These experiments demonstrate that D1V1-KLH can induce a stimulatory immune response without requirement for additional adjuvant when administered to human cord blood engrafted mice, suggesting that targeting through DC-SIGN stimulates DCs sufficiently to induce a productive immune response, and demonstrates that naïve responses and not only recall responses are provoked by targeting through DC-SIGN.

TABLE II
Number of DCs and T-cells recovered from grafted mice immunized with D1V1-KLH.
Cord blood grafted mice were immunized i.v. with either 100 μg D1V1-KLH or
100 μg 5G1.1-KLH. In addition, 2 mice were injected s.c. with 100 μg KLH
and Titermax. Spleens and lymph nodes were harvested 9 or 14 days later and
the frequency of T-cells and DCs was assessed by flow cytometry.
A. Nine days after immunization (data from spleen and lymph nodes)

| | | |
|---|---|---|
| D1V1-KLH | 5G1.1-KLH | KLH + Titermax |

| | #3254 SP | #3241 LN | #3241 SP | #3250 SP | #3250 LN |
|---|---|---|---|---|---|
| CD11c | 11.15 | 15.82 | 11.34 | 5.21 | 7.37 |
| CD4 | 10.77 | 11.24 | 10.43 | 7.51 | 11.3 |

B. Fourteen days after immunization with D1V1-KLH (#3239, 3247, 3255) or KLH and
Titermax (#3251) (data from spleen only)

| | mouse #3247 | mouse #3239 | mouse #3255 | mouse #3251 |
|---|---|---|---|---|
| CD11c | 9.24 | 5.02 | 13.89 | 9.38 |
| CD8 | 9.2 | 4.44 | 11.8 | 8.47 |
| CD4 | 4.88 | 4.2 | 9.83 | 9.55 |

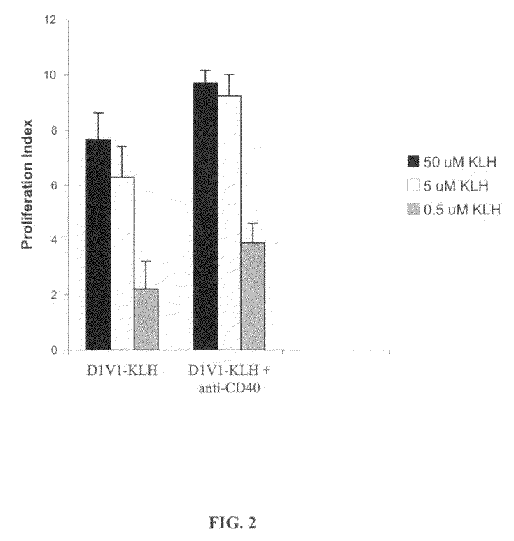

Anti-CD40 co-stimulation does not affect the overall proliferative response to KLH induced in human cord blood grafted mice immunized with D1V1-KLH. Although the magnitude of T cell proliferation to KLH after D1V1-KLH immunization was comparable to the magnitude of T cell proliferation after PHA stimulation already suggested that there is no requirement for additional adjuvant, concomitant administration of anti-CD40 and D1V1-KLH was evaluated for the ability to increase the KLH response. The ligation of CD40 by CD154 (CD40L) is a critical step in the interaction between APC and T cells (Howland, K. C., et al. 2000. J Immunol 164:4465-4470). Bonifaz et al. (J Exp Med 196:1627-1638 (2002)) demonstrated that simultaneous delivery of a DC maturation stimulus via CD40 was required for the elicitation of a stimulatory immune response with an anti-DEC-205:OVA construct. In the absence of co-stimulation, administration of anti-DEC-205:OVA induced tolerance. FIG. 2 shows that in contrast to targeting through DEC-205, antigen targeting through DC-SIGN is not substantially enhanced by anti-CD40 ligation. D1V1-KLH alone resulted in a T cell response to KLH similar to that achieved by a combination of D1V1-KLH and CD40 antibody.

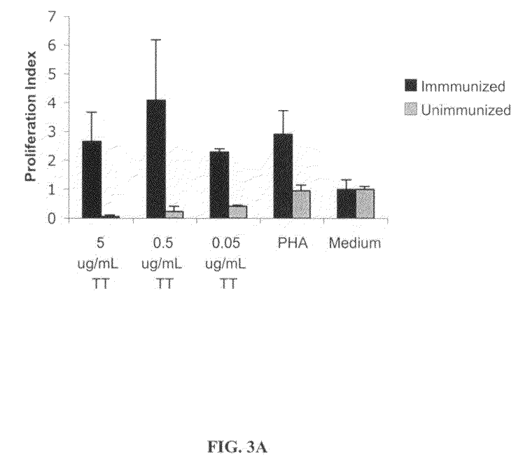

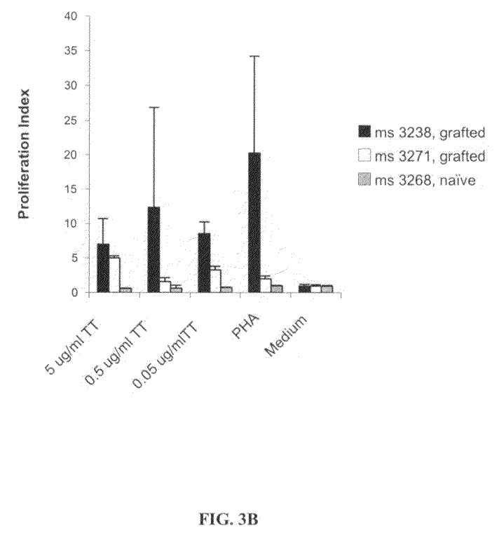

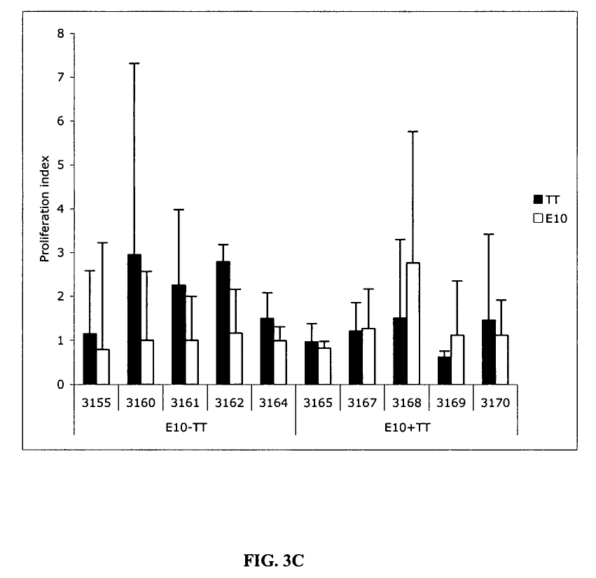

Induction of a proliferative response by in vivo antigen targeting through DC-SIGN is neither antigen specific nor limited to a particular anti-DC-SIGN antibody. In addition to KLH, it was evaluated whether an immune response to another model antigen, tetanus toxoid (TT), could be raised. Furthermore, it was addressed whether other DC-SIGN antibody constructs besides D1V1 can induce an immune response. Similar to the KLH experiments, it was first evaluated whether a human immune response could be raised to unconjugated TT in adjuvant in grafted mice. Splenocytes from mice that received one immunization strongly proliferated in response to TT, while unimmunized mice did not (FIG. 3A). The proliferative response to TT had the same magnitude as the proliferative response to PHA, indicating that a maximal response with TT was obtained. After a second immunization, the proliferative capacity of T cells in response to TT was 2-5 fold higher than after a single immunization. (FIG. 3B). However, in contrast to T cell responses, no antigen-specific immunoglobulin response was detectable. To raise an antibody response, further immunizations might be required, and most likely free antigen will have to be provided for uptake by B cells. Taken together, these experiments show that cord blood-grafted mice do mount a human T cell response to TT.

In further experiments, two different antibody conjugates targeting TT to DC-SIGN were tested. In contrast to the KLH antibody conjugate, TT constructs were genetically engineered to contain a TT epitope, instead of chemically linking the entire protein to the antibody. The TT epitope 632DR was expressed in the hinge region of the DC-SIGN reactive E10 antibody (E10-TT) (Dakappagari, N., et al. 2006. J Immunol 176:426-440). It was previously demonstrated that the E10-TT antigen conjugate, E10-632DR, can be processed and presented by human DCs in vitro, eliciting a significant proliferative response in T cells obtained from TT-vaccinated donors. To evaluate whether targeting of human DCs in vivo by E10-TT conjugate in Rag2^{-/-}γc^{-/-} reconstituted mice can evoke a naïve T cell response to TT, grafted mice were immunized with either E10-TT conjugate or with a mixture of E10 and TT. Nine days after immunization, three out of five mice immunized with the E10-TT conjugate evoked a 2-fold stronger immune response to TT than the five mice immunized with E10 and TT as a mixture (FIG. 3C). However, the overall proliferation indices were smaller than those observed with D1V1-KLH. In the same experiment, it was also evaluated whether a response against the antibody itself was provoked. Generally, this was not the case with the exception of mouse 3168 that had received a mixture of E10 and TT. Overall, this experiment confirms that antigen targeting through DC-SIGN can result in activation of naïve T cells, and is not limited to a specific antibody or antigen.

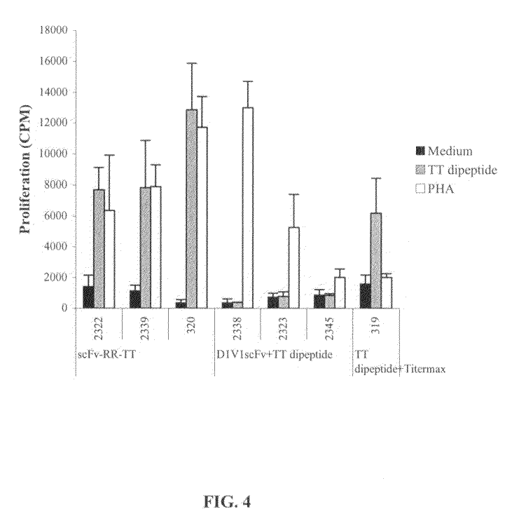

Induction of a proliferative T cell response by antigen targeting through DC-SIGN in vivo does not require dimerization. In the next set of experiments it was explored whether induction of a proliferative T cell response requires dimerization of DC-SIGN molecules. To evaluate whether a response can be evoked without dimerization, hD1V1-G2G4 was converted into a single chain antibody with V-regions separated by a 15 amino acid linker containing two copies of a TT epitope in tandem (designated, D1V1scFv-RR-TT). Proliferation of T cells in response to TT after immunization with TT dipeptide in adjuvant, D1V1scFv-RR-TT or D1V1scFv mixed with TT dipeptide was compared. Splenocytes from all three mice injected with D1V1scFv-RR-TT showed a strong proliferative response to the peptide comparable to PHA-stimulated cells. Unstimulated cells showed little proliferation (FIG. 4). None of the mice injected with the scFv and free dipeptide showed a response to TT, but successful engraftment of human T cells was demonstrated by proliferation of cells to PHA. The mouse that received TT dipeptide in Titermax also showed a proliferative response to TT, although surprisingly not to PHA. These data indicate that even antigen targeting with a single chain antibody to DC-SIGN that should be primarily in monomeric form induces a stimulatory immune response, and no dimerization of DC-SIGN molecules appears to be required.

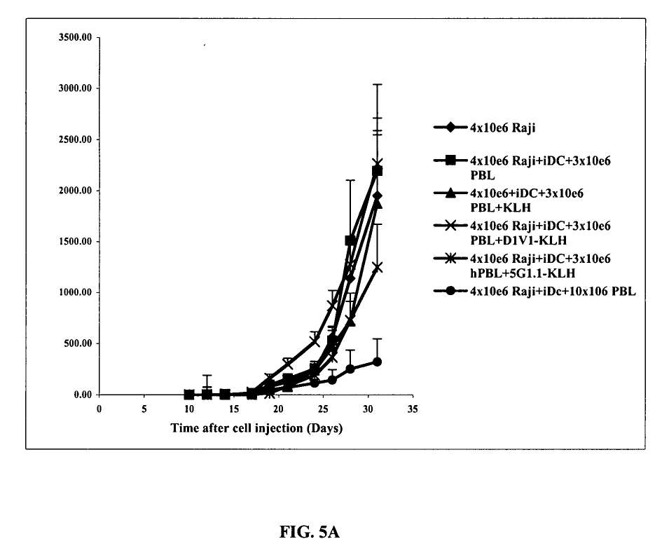

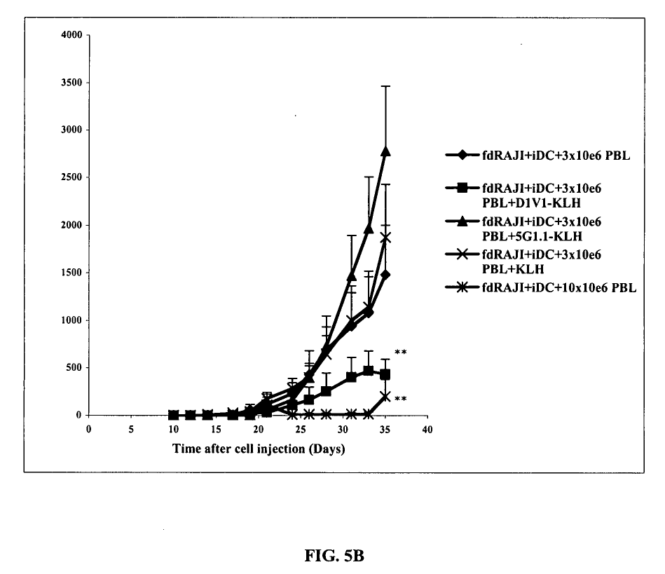

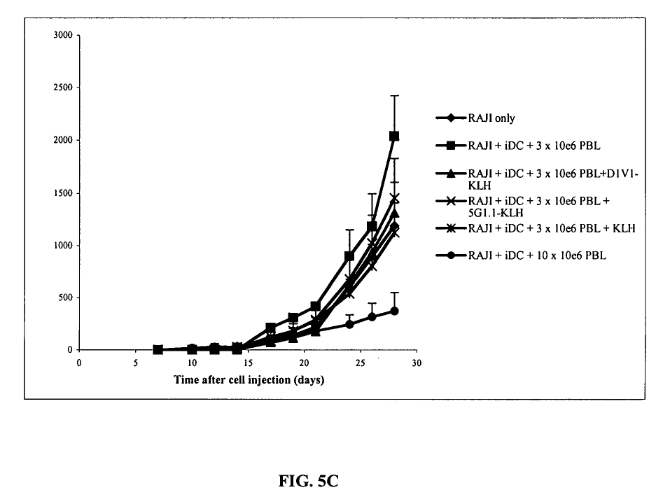

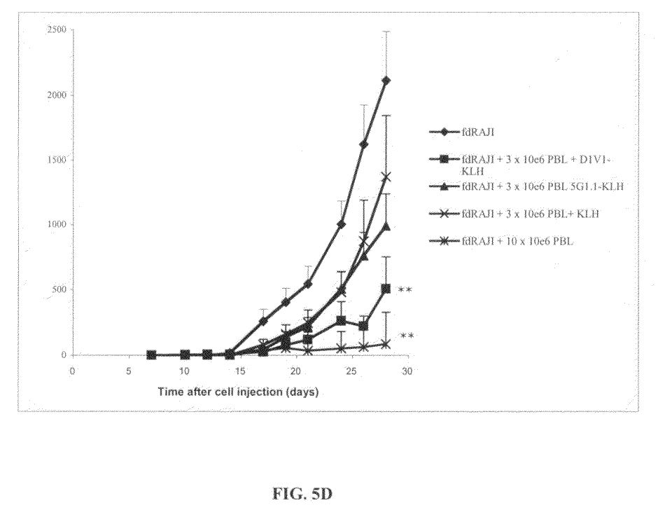

D1V1-KLH as potential adjuvant in cancer therapy. The ultimate goal of antigen targeting through DC-SIGN is to direct the immune system, for example, against cancer or infectious disease antigens, resulting in the eradication of cancer cells or infectious agents. In all of the experiments using cord blood grafted mice, proliferation of T cells was the read-out for a stimulatory immune response. It was next addressed whether antigen targeting through DC-SIGN could result in inhibition of tumor growth. Again, D1V1-KLH was used as a model system. It has been reported that addition of KLH to cancer vaccines containing DCs loaded with peptides or tumor lysates increased vaccine efficacy in clinical trials (Ragupathi, G., et al. 2003. Clin Cancer Res 9:5214-5220; Krug, L. M., et al. 2004. Clin Cancer Res 10:6094-6100; Slovin, S. F. 2005. Clin Prostate Cancer 4:118-123). Therefore, it was addressed whether linkage of KLH to hD1V1 (D1V1-KLH) can provide enhanced adjuvant properties, resulting in increased tumor cell killing by the immune system. A model suitable to evaluate the effect of human immune cells on human tumor growth in immune deficient mice (NOD/SCID) was previously established. In this model, subcutaneous injection of the Burkitt lymphoma cell line RAJI together with 0-5×10⁶ hPBL and 30,000 DCs does not inhibit tumor growth, but administration of more than 5×10⁶ hPBLs significantly inhibits tumor growth. In this model, it was evaluated whether administration of D1V1-KLH with 30,000 DCs and 3×10⁶ hPBLs results in tumor growth inhibition. Tumor growth in D1V1-KLH treated groups was compared to tumor growth in groups treated with the control antibody construct 5G1.1-KLH or KLH alone. As expected, 3 million hPBLs with 30,000 immature human DCs did not significantly inhibit the tumor growth of RAJI cells (FIG. 5A), while 10 million hPBLs with 30,000 immature DCs inhibit tumor growth by 90%. Administration of D1V1-KLH inhibited tumor growth by 50%, while the control antibody 5G1.1-KLH or KLH alone did not affect tumor growth in this experiment.

It was further evaluated whether the presence of apoptotic cells could potentially increase presentation of RAJI-derived antigen, and result in a stronger tumor growth inhibition in the presence of D1V1-KLH in this tumor model. Indeed, when mice were injected with a mixture of fludarabine-treated and untreated RAJI cells, tumor growth was inhibited by 50% in 5G1.1-KLH and KLH-treated groups, and by 75% in the D1V1-KLH-treated group (FIG. 5B). The observed tumor growth inhibition by KLH-hD1V1 G2/G4 was significant from day 31 after cell injection (p<0.05) until the end of the study. When this experiment was repeated with the same donor, no significant tumor growth inhibition was observed in mice injected with RAJI cells alone with any of the treatments tested (FIG. 5C), however, in mice that had received a mixture of fludarabine and untreated cells, treatment with D1V1-KLH resulted in approximately 80% tumor growth inhibition (FIG. 5D).

These experiments suggest that D1V1-KLH administration results in enhanced immune stimulation compared to administration of KLH or KLH-5G1.1, mediating increased tumor cell killing. D1V1-KLH might be therapeutically useful as an adjuvant combined with tumor peptides or other forms of tumor antigen or any therapy resulting in tumor cell lysis such as radiation or chemotherapy. Furthermore, these experiments provide a proof of principle that DC-SIGN antibodies engineered to contain tumor or infectious disease antigens can potentially raise an enhanced immune response compared to non-targeted immunization approaches.

### EQUIVALENTS

The present invention provides among other things compositions and methods for enhancing an adjuvant effect. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: Leach, D. R., et al. 1996. Science 271:1734-1736; Maker, A. V., et al. 2005. Ann Surg Oncol 12:1005-1016; Takahashi, T., Y. et al. 1998. Int Immunol 10:1969-1980; Dannull, J., et al. 2005. J Clin Invest 115:3623-3633; Schreurs, M. W., et al. 2000. Crit Rev Oncog 11: 1-17; Caparros, E., et al. 2006. Blood. 107:3950-8; Croce, M. V., et al. 2002. Drugs Today (Barc) 38:759-768; Millard, A. L., et al. 2003. Vaccine 21:869-876; Mahnke, K., et al. 2005. Cancer Res 65:7007-7012; Kato, M., et al. 2006. Int Immunol; and Mueller, J. P., et al. 1997. Mol Immunol 34:441-452.

1. A method for enhancing the effect of an adjuvant, comprising administering to an animal an agent comprising an adjuvant attached to a compound that binds to a cell surface marker of an antigen presenting cell (APC), thereby targeting the adjuvant to an antigen presenting cell (APC) and enhancing the effect of the adjuvant by stimulating an immune response to an antigen other than the compound or the adjuvant.

2. The method of claim 1, wherein said adjuvant is attached to said compound by a covalent bond.

3. The method of claim 1, wherein said compound is an antibody that binds to a cell surface marker of an APC.

4. The method of claim 3, wherein said antibody is an antigen binding fragment of an antibody.

5. The method of claim 3, wherein said antibody is an internalizing antibody.

6. The method of claim 3, wherein said antibody is a humanized antibody.

7. The method of claim 1, wherein said cell surface marker is an internalizing receptor.

8. The method of claim 1, wherein said APC is a dendritic cell.

9. The method of claim 8, wherein said cell surface marker is a C-type lectin.

10. The method of claim 8, wherein said cell surface marker is DC-SIGN.

11. The method of claim 10, wherein said compound that binds to DC-SIGN is a mannose carbohydrate, a fucose carbohydrate, a plant lectin, an antibiotic, a sugar, a protein, or an antibody.

12. The method of claim 11, wherein said compound that binds to DC-SIGN is an antibody.

13. The method of claim 12, wherein said antibody is a monoclonal antibody.

14. The method of claim 1, wherein said adjuvant is a mineral salt, a small molecule, a saponin, a polysaccharide, a lipid, a nucleic acid, a protein or a peptide.

15. The method of claim 14, wherein said adjuvant is a protein.

16. The method of claim 14, wherein said adjuvant is not a lipid.

17. The method of claim 15, wherein said protein is keyhole limpet hemocyanin (KLH) or bacillus calmette guerin (BCG).

18. The method of claim 1, wherein said animal is a human.

19. The method of claim 1, wherein said adjuvant stimulates a naive immune response.

20. The method of claim 1, wherein said animal was not previously vaccinated with said adjuvant.

21. The method of claim 1, wherein said agent is formulated in a pharmaceutical composition.

22. The method of claim 21, wherein said pharmaceutical composition further comprises antigen.

23. The method of claim 1, wherein said agent further comprises an antigen attached to said compound.

24. The method of claim 23, wherein said antigen is covalently attached to said compound.

25. The method of claim 23, wherein said agent is formulated in a pharmaceutical composition.

26. The method of claim 25, wherein said pharmaceutical composition further comprises antigen which is not attached to said compound.

27. The method of claim 26, wherein the antigen attached to the compound and the antigen which is not attached to the compound are the same.

28. The method of claim 23, wherein said antigen is not a nucleic acid vaccine.

29. The method of claim 23, wherein said antigen is a protein or a peptide.

30. A method for stimulating an immune response in an animal in need thereof, comprising administering to said animal an agent comprising an adjuvant attached to a compound that binds to a cell surface marker of an antigen presenting cell (APC), wherein said agent stimulates an immune response to an antigen other than the compound or the adjuvant.

31-60. (canceled)

61. A method for stimulating an immune response in an animal, comprising administering to said animal an APC targeting agent comprising a DC-SIGN specific antibody covalently linked to an adjuvant, wherein said APC targeting agent stimulates an immune response to an antigen other than the DC-SIGN antibody or the adjuvant, and wherein said animal was not previously vaccinated with said adjuvant.

62. An immunostimulatory agent comprising a compound that binds to a cell surface marker of an antigen presenting cell (APC), an adjuvant and an antigen, wherein the compound, adjuvant and antigen are attached.

63-90. (canceled)

91. An immunostimulatory composition comprising a compound that binds to a cell surface marker of an antigen presenting cell (APC), an adjuvant and an antigen, wherein at least one of the adjuvant or the antigen is attached to said compound.

92-117. (canceled)
